(19) United States
(12) Patent Application Publication
(10) Pub. No.: US 2026/0242508 A1
(43) Pub. Date: Aug. 20, 2026

(54) ANTIBODIES TO NEOANTIGENS AND USES THEREOF

(71) Applicant: Moonshot Antibodies, LLC, Shelburne, VT (US)

(72) Inventors: David N. Krag, Shelburne, VT (US); Girja S. Shukla, South Burlington, VT (US); Stephanie C. Fournier, Colchester, VT (US); Sunny Yujing Sun, South Burlington, VT (US)

(21) Appl. No.: 19/305,419

(22) Filed: Aug. 20, 2025

Related U.S. Application Data
(63) Continuation of application No. 17/433,982, filed on Feb. 26, 2020.
(60) Provisional application No. 62/810,591, filed on Feb. 26, 2019.
(60) Provisional application No. 62/855,819, filed on May 31, 2019.

(51) Int. Cl. C07K 16/30; A61K 31/437; A61K 35/17; A61K 39/00; A61K 39/395; A61K 40/10; A61K 40/42; A61P 35/00; C07K 16/28

(52) U.S. Cl. CPC C07K 16/3053; A61K 31/437; A61K 35/17; A61K 39/39558; A61K 40/10; A61K 40/4271; A61P 35/00; C07K 16/2818; A61K 2039/507; A61K 2239/31; A61K 2239/38; A61K 2239/57; C07K 2317/92

(57) ABSTRACT

Compositions and methods involving anti-neoantigen antibodies and/or effector cells armed with anti-neoantigen antibodies are disclosed herein. Such compositions may also include immune checkpoint inhibitors and may be used, for example, in the treatment of cancers.

### RELATED APPLICATIONS

This application claims the benefit under 35 U.S.C. § 119(e) of U.S. provisional application No. 62/810,591, filed Feb. 26, 2019, and U.S. provisional application No. 62/855,819, filed May 31, 2019, the entire disclosure of each of which is incorporated herein by reference in its entirety.

### BACKGROUND OF THE INVENTION

Immunology is based on distinguishing self from non-self. Most pathogens contain molecular signatures that are recognized by the host, resulting in immune responses. However, in cancer, such molecular signatures are generally not expressed by tumor cells, so host immune responses may be limited. A subset of immune cells, T cells, can recognize tumor antigens expressed by tumor cells. Further, tumor-specific neoantigens, which contain non-synonymous somatic mutations and may be processed and presented on the cell surface of tumors, are sufficiently different from those found on noncancerous cells, that they may be recognized as foreign by neoantigen-specific T cells. Since noncancerous cells do not display neoantigens, neoantigen-specific T cells are not subjected to central or peripheral tolerance mechanisms and do not induce the destruction of normal tissue, and can therefore cause T cell-mediated destruction of the tumor cells.

T cell therapy has been examined, and protocols have been developed to culture and expand the cells ex vivo¹. These advancements led to adoptive cell therapy using tumor-infiltrating lymphocytes (TILs)². Dramatic responses in a minority of patients demonstrated the powerful capacity of T cells to inhibit tumors³. However, the final cellular reagents in patients are not readily controlled. The final product delivered to the patient is an uncontrolled mix of T cell clones binding to unspecified targets. In many patients, TILs are not recoverable⁴.

Chimeric antigen receptor T cell therapy (CAR-T) was then designed to gain greater control over the final product delivered to the patient. In CAR-T cell therapy, a binding element is introduced to T cells to direct therapy to a specific target. Unlike TIL therapy, CAR-T cell therapy is largely limited to a single targets. As a result, this therapy does not address the universal problem of tumor heterogeneity. Tumor cell heterogeneity prevents a single antibody from binding sufficiently to all cancer cells in the tumor population. For example, Herceptin invariably cannot bind to and mediate cell death of all cancer cells in a tumor because of variable expression of HER2. As a result, Herceptin does not cure stage IV patients. CAR-T cell therapy faces the same limitation as antibodies like Herceptin. In addition, targeting tumor-specific antigens by CAR-T cell therapy appears to not be possible and in most trials, the target is limited to tissue-specific targets such as CD19⁶. Furthermore, targeting CD19 or any other tissue-specific target will mediate cell death of normal cells along with tumor cells.

### SUMMARY OF THE INVENTION

The disclosure, in some aspects, provides a method of treating a cancer in a subject, the method comprising administering to a subject having cancer, effector cells comprising two or more anti-neoantigen antibodies in an effective amount to treat the cancer. In some embodiments, the method further comprises administering an immune checkpoint inhibitor to the subject having cancer. In some embodiments, the two or more anti-neoantigen antibodies comprise at least three, at least four, at least five, at least 6, at least 7, at least 8, at least 9, or at least 10 anti-neoantigen antibodies.

In some embodiments, the effector cells are syngeneic donor effector cells. In some embodiments, the syngeneic donor effector cells are selected from the group consisting of natural killer (NK) cells, neutrophils, T cells, B cells, monocytes/macrophages, and combinations thereof.

In some embodiments, the effector cells and the immune checkpoint inhibitor are administered simultaneously. In some embodiments, the effector cells are administered prior to administration of the immune checkpoint inhibitor. In some embodiments, the effector cells are administered to the subject at least twice. In some embodiments, the effector cells are administered to the subject five times. In some embodiments, the effector cells are administered via intratumoral injection. In some embodiments, the effector cells are administered intravenously. In some embodiments, the immune checkpoint inhibitor is administered via intraperitoneal injection or intravenous injection or subcutaneous injection.

In some embodiments, the cancer is selected from the group consisting of basal cell carcinoma, bladder cancer, bone cancer, bowel carcinoma, breast cancer, carcinoid, anal squamous cell carcinoma, castration-resistant prostate cancer (CRPC), cervical carcinoma, colorectal cancer (CRC), colon cancer cutaneous squamous cell carcinoma, endometrial cancer, esophageal cancer, gastric carcinoma, gastroesophageal junction cancer, glioblastoma/mixed glioma, glioma, head and neck cancer, hepatocellular carcinoma, hematologic malignancy. liver cancer, lung cancer, melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer, rectal carcinoma, renal cancer, sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, or uterine sarcoma.

In some embodiments, the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

In some embodiments, the immune checkpoint inhibitor is administered on a schedule of one dose every 7-30 days; one dose every 14 days; or one dose every 21 days. In one embodiment, the anti-CTLA-4 antibody is ipilimumab, and optionally is administered at a dose of about 3 mg/kg-10 mg/kg or a fixed dose of about 240 mg-800 mg. In one embodiment, the anti-PD1 antibody is pembrolizumab, and optionally is administered at a dose of about 3 mg/kg-10 mg/kg or a fixed dose of about 240 mg-800 mg. In some embodiments, the immune checkpoint inhibitor is selected from the group consisting of pembrolizumab, nivolumab, J43, RMP1-14, atezolizumab, ipilimumab, and combinations thereof.

In some embodiments, the effector cells and, optionally, the immune checkpoint inhibitor produce a significant reduction in tumor volume relative to administration of effector cells without anti-neoantigen antibodies or immune checkpoint inhibitor alone.

In some embodiments, the effector cells and, optionally, the immune checkpoint inhibitor produce a significant reduction in tumor volume.

In some embodiments, the effector cells and, optionally, the immune checkpoint inhibitor produce a significant increase in survival rate relative to administration of effector cells without anti-neoantigen antibodies or immune checkpoint inhibitor alone.

In some embodiments, the effector cells and, optionally, the immune checkpoint inhibitor produce a significant increase in survival rate.

In some embodiments, the effector cells and, optionally, the immune checkpoint inhibitor produce a durable immune response relative to administration of effector cells without anti-neoantigen antibodies or immune checkpoint inhibitor alone. In some embodiments, the durable immune response lasts for at least six months.

In some embodiments, the method further comprises administering an anti-cancer agent. In some embodiments, the anti-cancer agent is selected from the group consisting of cancer vaccine, chemotherapy, radiation, and immunotherapeutic. In one embodiment, the immunotherapeutic is a modified T cell. In some embodiments, the anti-cancer agent is a B-RAF inhibitor. In some embodiments, the B-RAF inhibitor is vemurafenib.

In some embodiments, the subject is non-responsive to the immune checkpoint therapy.

The disclosure, in another aspect, provides a composition comprising effector cells comprising two or more anti-neoantigen antibodies and a pharmaceutically acceptable carrier. In some embodiments, the composition comprises at least three, at least four, at least five, at least 6, at least 7, at least 8, at least 9, or at least 10 anti-neoantigen antibodies.

In some embodiments, the effector cells are syngeneic donor effector cells. In some embodiments, the syngeneic donor effector cells are selected from the group consisting of natural killer (NK) cells, neutrophils, T cells, B cells, monocytes/macrophages, and combinations thereof.

In some embodiments, the anti-neoantigen antibodies are directed to neoantigens related to the cell surface of a tumor or secretory proteins. In some embodiments, the anti-neoantigen antibodies are each directed to a different neoantigen. In some embodiments, the neoantigens each comprise a single amino acid substitution.

In some embodiments, the two or more anti-neoantigen antibodies are present in the composition in equal concentrations. In some embodiments, the composition comprises nine anti-neoantigen antibodies. In some embodiments, the composition comprises four anti-neoantigen antibodies.

In some embodiments, the composition further comprises an immune checkpoint inhibitor. In some embodiments, the immune checkpoint inhibitor is a PD1 inhibitor. In some embodiments, the PD1 inhibitor is an anti-PD1 antibody.

In some embodiments, the anti-neoantigen antibodies are monoclonal antibodies. In some embodiments, the anti-neoantigen antibodies are polyclonal antibodies. In some embodiments, the polyclonal antibodies are human antibodies. In some embodiments, the anti-neoantigen antibodies are pooled human antibodies.

The disclosure, in another aspect, relates to a method of treating cancer in a subject, the method comprising administering to a subject having cancer, two or more anti-neoantigen antibodies in an effective amount to treat the cancer. In a further embodiment, the method further comprises administering an immune checkpoint inhibitor to the subject having cancer.

In some embodiments, the two or more anti-neoantigen antibodies comprise at least three, at least four, at least five, at least 6, at least 7, at least 8, at least 9, or at least 10 anti-neoantigen antibodies. In some embodiments, at least one of the two or more anti-neoantigen antibodies is in a chimeric antigen receptor (CAR) format, while the at least one other anti-neoantigen antibody is an antibody.

In some embodiments, the two more anti-neoantigen antibodies and the immune checkpoint inhibitor are administered simultaneously. In another embodiment, the two or more anti-neoantigen antibodies are administered prior to administration of the immune checkpoint inhibitor.

In some embodiments, the two more anti-neoantigen antibodies are administered to the subject at least twice. In some embodiments, the two more anti-neoantigen antibodies are administered to the subject four times.

In one embodiment, the two or more anti-neoantigen antibodies are administered via intratumoral injection. In another embodiment, the immune checkpoint inhibitor is administered via intraperitoneal injection. In one embodiment, the two or more anti-neoantigen antibodies are administered via intravenous injection.

In some embodiments, the cancer is selected from the group consisting of basal cell carcinoma, bladder cancer, bone cancer, bowel carcinoma, breast cancer, carcinoid, anal squamous cell carcinoma, castration-resistant prostate cancer (CRPC), cervical carcinoma, colorectal cancer (CRC), colon cancer cutaneous squamous cell carcinoma, endometrial cancer, esophageal cancer, gastric carcinoma, gastroesophageal junction cancer, glioblastoma/mixed glioma, glioma, head and neck cancer, hepatocellular carcinoma, hematologic malignancy. liver cancer, lung cancer, melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer, rectal carcinoma, renal cancer, sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, or uterine sarcoma.

In some embodiments, the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

In some embodiments, the immune checkpoint inhibitor is administered on a schedule of one dose every 7-30 days; one dose every 14 days; or one dose every 21 days. In some embodiments, the anti-CTLA-4 antibody is ipilimumab, and optionally is administered at a dose of about 3 mg/kg-10 mg/kg or a fixed dose of about 240 mg-800 mg. In some embodiments, the anti PD-1 antibody is pembrolizumab and optionally is administered at a dose of about 3 mg/kg-10 mg/kg or a fixed dose of about 240 mg-800 mg.

In some embodiments, the immune checkpoint inhibitor is selected from the group consisting of pembrolizumab, nivolumab, J43, RMP1-14, atezolizumab, ipilimumab, and combinations thereof.

In some embodiments, the two or more anti-neoantigen antibodies and the immune checkpoint inhibitor produce a significant reduction in tumor volume relative to administration of the antibodies or immune checkpoint inhibitor alone. In some embodiments, the two or more anti-neoantigen antibodies and the immune checkpoint inhibitor produce a significant reduction in tumor volume.

In some embodiments, the two or more anti-neoantigen antibodies and the immune checkpoint inhibitor produce a significant increase in survival rate relative to administration of the antibodies or immune checkpoint inhibitor alone. In some embodiments, the two or more anti-neoantigen antibodies and the immune checkpoint inhibitor produce a significant increase in survival rate.

In some embodiments, the effector cells and, optionally, the immune checkpoint inhibitor produce a durable immune response relative to administration of effector cells without anti-neoantigen antibodies or immune checkpoint inhibitor alone. In some embodiments, the durable immune response lasts for at least six months.

In some embodiments, the method further comprises administering an anti-cancer agent. In some embodiments, the anti-cancer agent is selected form the group consisting of cancer vaccine, chemotherapy, radiation, and immunotherapeutic. In some embodiments, the immunotherapeutic is a modified T cell. In some embodiments, the anti-cancer agent is a B-RAF inhibitor. In one embodiment, the B-RAF inhibitor is vemurafenib.

In some embodiments, the subject is non-responsive to the immune checkpoint therapy. In one embodiment, the subject does not have a favorable response to the immune checkpoint therapy based on RECIST (Response Evaluation Criteria In Solid Tumors), or irRECIST (Immune-related Response Evaluation Criteria In Solid Tumors), or iRECIST.

In some embodiments, the two or more anti-neoantigen antibodies are administered in separate formulations to the subject. In some embodiments, the two or more anti-neoantigen antibodies are administered in the same formulation to the subject.

In some embodiments, the formulation of two or more anti-neoantigen antibodies comprises at least two, at least three, at least four, at least five, at least 6, at least 7, at least 8, at least 9, or at least 10 anti-neoantigen antibodies.

The disclosure, in another aspect, provides a composition comprising two or more anti-neoantigen antibodies and a pharmaceutically acceptable carrier. In some embodiments, the composition further comprises at least three, at least four, at least five, at least 6, at least 7, at least 8, at least 9, or at least 10 anti-neoantigen antibodies.

In some embodiments, the composition comprises a CAR T cell, wherein at least one of the anti-neoantigen antibodies is in the form of the CAR T cell. In some embodiments, the CAR T cell comprises at least two different anti-neoantigen antibodies, each different anti-neoantigen antibody directed to a different neoantigen. In some embodiments, the composition comprises at least one anti-neoantigen antibody in the form of a CAR T cell and at least one anti-neoantigen antibody in the form of an antibody.

In some embodiments, the anti-neoantigen antibodies are directed to neoantigens related to the cell surface of a tumor or secretory proteins. In some embodiments, the anti-neoantigen antibodies are each directed to a different neoantigen. In some embodiments, the neoantigens each comprise a single amino acid substitution. In some embodiments, the two or more anti-neoantigen antibodies are present in the composition in equal concentrations. In one embodiment, the composition comprises nine anti-neoantigen antibodies.

In some embodiments, the composition further comprises an immune checkpoint inhibitor. In one embodiment, the immune checkpoint inhibitor is a PD1 inhibitor. In some embodiments, the PD1 inhibitor is an anti-PD1 antibody. In one embodiment, the anti-neoantigen antibodies are monoclonal antibodies. In some embodiments, the anti-neoantigen antibodies are polyclonal antibodies. In some embodiments, the polyclonal antibodies are human antibodies. In one embodiment, the anti-neoantigen antibodies are pooled human antibodies. In some embodiments, the anti-neoantigen antibodies are rabbit antibodies. In some embodiments the anti-neoantigen antibodies are humanized antibodies.

Another aspect of the disclosure provides a method of treating a cancer in a subject, the method comprising: screening a tumor biopsy from the subject, identifying, based on the results of the screen, two or more neoantigens for targeted treatment, and administering to the subject having cancer, the two or more anti-neoantigen antibodies identified or an effector cell comprising the two or more anti-neoantigen antibodies identified, in an effective amount to treat the cancer.

Each of the limitations of the invention can encompass various embodiments of the invention. It is, therefore, anticipated that each of the limitations of the invention involving any one element or combinations of elements can be included in each aspect of the invention. This invention is not limited in its application to the details of construction and the arrangement of components set forth in the following description or illustrated in the drawings. The invention is capable of other embodiments and of being practiced or of being carried out in various ways. The details of one or more embodiments of the invention are set forth in the accompanying Detailed Description, Examples, Claims, and Figures. Other features, objects, and advantages of the invention will be apparent from the description and from the claims.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 shows an assessment of the rabbit polyclonal antibodies raised against selected mutated peptides described in the Examples. Binding curves of serially diluted (2-fold) samples of individual rabbit anti-peptide antibodies are shown. Each absorbance value plotted for different antibody samples represents average values from duplicate (≤5%) wells.

FIG. 2 depicts the effects of different treatments on tumor growth in a mouse B16-F10 melanoma model. The values represent mean±SEM of six or surviving animals in each group.

FIG. 3 illustrates the effects of different treatments, including antibodies with a PD1 checkpoint inhibitor, on tumor growth in a mouse B16-F10 melanoma model. Tukey's multiple comparisons test determined that both the antibody cocktail and PD1i-treated groups were significantly different (P≤0.05) in comparison to PBS and/or respective Ig control groups. The values represent mean±SEM of six or surviving animals in each group.

FIG. 4 depicts the effects of different treatments on animal survival in a mouse B16-F10 melanoma model.

FIG. 5 illustrates the effects of different treatments, including antibodies with a PD1 checkpoint inhibitor, on animal survival in a mouse B16-F10 melanoma model. DPI, days post tumor implant.

FIG. 6 shows a binding analysis of affinity-enriched rabbit polyclonal antibodies raised against selected mutated peptides to mouse B16-F10 tumor sections by immunofluorescence microscopy. The staining by a cocktail of nine antibodies presented in the far-left panel shows strong uniform binding to the tumor tissue. Considerable variations in the staining by nine individual antibodies are seen in other panels labeled by the names of target mutated proteins. The lower right corner panel represents negative control showing no tumor binding by normal rabbit IgG and fluorescent secondary antibody.

FIG. 7 is four photos showing representative mice at 12 days post-tumor implantation. Panel A, PBS control; panel B, PD1i, panel C, single dose antibody cocktail+Pd1i, and panel D, 4 doses of antibody cocktail+PD1i.

FIG. 8 shows the effect of different treatments on survival of mice implanted with B16-F10 melanoma cells. The combined treatment with effector cells armed with nine antibody cocktail and PD1i increased the survival of mice. EC=Effector cells; EC-armed with Ab Cocktail=Effector cells-armed with a cocktail of all the 9 antibodies against selected mutated peptides; TCT=Tumor cell transplantation; T1-T5=EC Treatment days; DPI=Days post-implantation.

FIGS. 9A-9E are graphs showing tumor volume seven days after implantation (FIG. 9A), eight days post-implantation (FIG. 9B), nine days post-implantation (FIG. 9C), 10 days post-implantation (FIG. 9D), and 13 days post-implantation (FIG. 9E). Mice were injected with the indicated treatments on days 3, 6, 8, 10, 13, and 15 days post-implantation (DPI).

FIG. 10 shows the effect of different treatments on B16-F10 tumor growth in mice. Significant tumor growth retardations (34% to 49%) were observed in the mice treated with effector cells armed with the nine antibody cocktail and PD1i, as compared to PBS control group. Two-way ANOVA analysis of the data shows significantly different curves by treatment and time. *Significantly different (P≤0.05) in comparison to PBS and PD1i alone groups as determined by Tukey's multiple comparisons test. The values represent mean±SEM of six mice in each group. EC=Effector cells; EC-armed with Ab Cocktail=Effector cells-armed with a cocktail of all the 9 rabbit antibodies against selected mutated peptides; TCT=Tumor cell transplantation; T1-T5=EC Treatment days; DPI=Days post-implantation.

FIGS. 11A-11E are graphs showing tumor volume seven days after implantation (FIG. 11A), 10 days post-implantation (FIG. 11B), 11 days post-implantation (FIG. 11C), 12 days post-implantation (FIG. 11D), and 13 days post-implantation (FIG. 11E). Mice were injected with the nine antibody cocktail or the four highest binders (HB) antibody cocktail on days 3, 4, 5, 6, and 7 days post-implantation (DPI). The mice were injected with the anti-PD1 antibody on days 3, 5, 7, 10, and 12 DPI.

FIG. 12 shows the effect of combined treatment of PD1i and effector cells armed with either a cocktail of nine antibodies or four high-binding antibodies on tumor growth in mice implanted with B16-F10 melanoma cells. Significant tumor growth retardations (44% to 68%) were observed in the mice treated with effector cells armed with 9 antibody cocktail and PD1i, as compared to PBS control group. A more robust growth retardation (64% to 87%) was observed in the group that received combined treatment of effector cells armed with the cocktail of 4 high-binding antibodies and PD1i. Two-way ANOVA analysis of the data shows significantly different curves by treatment and time. *Significantly different (P<0.05) in comparison to PBS and PD1i alone groups as determined by Turkey's multiple comparisons test. The values represent mean±SEM of six mice in each group. EC-armed with 9-Ab Cocktail=Effector cells-armed with a cocktail of all the 9 rabbit anti-antibodies against selected mutated peptides; EC-4HB-Ab Cocktail=Effector cells-armed with a cocktail of 4 high tumor-binding rabbit antibodies; TCT=Tumor cell transplantation; T1-T5=EC Treatment days; DPI=Days post-implantation.

FIG. 13 shows representative density plots and histograms from flow cytometric analyses of binding by a cocktail of nine rabbit antibodies to spleen leukocytes. Leukocyte samples were gated to include most of the live cells based on side (SSA) and forward (FSA) scatter characteristics. The histograms showing the gated fluorescently stained and unstained cell populations, represent the cells bound (Ig⁺) to and not bound (Ig⁻) to rabbit antibodies.

FIG. 14 shows the effect of treatment with a cocktail of four high tumor-binding antibodies on survival of mice implanted with B16-F10 melanoma cells. The combined treatment with effector cells-armed with 4 high tumor-binding antibody cocktail and PD1i increased the survival of mice longer (31 d) than those treated with the effector cells armed with a cocktail of all the 9 antibodies and PD1i (22.5 d). EC=Effector cells; EC-armed with 9-Ab Cocktail=Effector cells-armed with a cocktail of all the 9 rabbit antibodies against selected mutated peptides; EC-4HB-Ab Cocktail=Effector cells-armed with a cocktail of 4 high tumor-binding rabbit antibodies; TCT=Tumor cell transplantation; TCT=Tumor cell transplantation; T1-T5=EC Treatment days; DPI=Days post-implantation.

### DETAILED DESCRIPTION OF THE INVENTION

The present disclosure relates, in one aspect, to the surprising discovery that cocktails of neoantigen antibodies have significant anti-tumor effects. By using multiple antibodies that bind multiple tumor-specific antigens, it has been found to result in dense homogeneous binding of all of the cancer cells. Antibodies are highly bioactive and readily induce cancer cell cytotoxicity as long as antibody binding achieves a critical threshold. In clinical practice, however, monoclonal antibodies never functionally achieve critical threshold binding on the full population of heterogeneous cancer cells. Without wishing to be bound by theory, targeting the unique set of tumor-specific mutations present in a patient's cancer by using multiple antibodies is thought to overcome the limitation of single-target monoclonal antibodies. The data presented herein illustrates that pooling multiple anti-neoantigen antibodies results in intense and homogenous antibody binding across the cell population. Significant inhibition of tumor growth was observed with the pooled antibody treatment. Even a single treatment inhibited growth of an aggressive tumor in mice, while four treatments prevented tumor growth in 50% of the mice. Further, the response was durable and rendered the responding mice resistant to subsequent tumor challenge.

Therefore, the present disclosure relates, in one aspect, to cocktails of neoantigen antibodies having significant anti-tumor effects. As described herein, multiple neoantigen antibody cocktails were found to significantly reduce tumor volume and significantly increase subject survival relative to controls (untreated or administration of an isotype antibody). Many tumors exhibit increased checkpoint activity; however, only a minority of patients show durable complete responses following administration of checkpoint inhibitors. As disclosed herein, the multiple neoantigen antibody cocktail, when administered with a checkpoint inhibitor (e.g., an anti-PD1 antibody), significantly reduced tumor volume and increased subject survival relative to controls (including administration of the anti-PD1 antibody alone).

The present disclosure also provides methods of cell therapy using a cocktail of neoantigen antibodies to redirect multiple types of immune effector cells to multiple tumor-specific targets. Prior to delivery, effector cells may be coated ex vivo with multiple tumor-specific antibodies (e.g., a cocktails of neoantigen antibodies, as described herein). Without wishing to be bound by theory, it is thought that simultaneously targeting multiple tumor-specific antigens may overcome the fundamental problem of tumor heterogeneity, which prevents a single antibody from binding sufficiently to all cancer cells in the tumor populations. Incubating effector cells ex vivo vastly reduces the amount of antibody required to cause tumor inhibition. Unlike conventional T cell therapy, expansion of effector cells ex vivo is not required.

As demonstrated herein, ex vivo arming a mixed population of immune effector cells with antibodies targeting multiple tumor-specific mutated proteins in conjunction with PD1 inhibition delays tumor growth and prolongs survival in a subject having a highly aggressive melanoma. Combining a mixture of 9 antibodies (a multiple neoantigen antibody cocktail) resulted in dense homogeneous binding to histological sections of melanoma.

Tumor inhibition was greater if the armed effector cells were given daily instead of every 2-3 days. This is consistent with the expected short duration of antibodies bound to effector cells. Rapid off kinetics provides built-in safety for rapid termination of effector cell activity. It appears that the types of tumor-specific mutations targeted in the studies described herein are common and abundant in multiple types of human cancers.

Accordingly, the neoantigen antibody cocktails and checkpoint inhibitors and/or armed effector cells described herein may be used to treat subjects who have cancer, in particular, those subjects having cancer that is non-responsive to checkpoint inhibitor monotherapy.

Anti-neoantigen antibodies are generated after vaccination with short neoantigen peptides. Neoantigens, or tumor-specific antigens, are antigens that are present in one or more tumor cells, but that are not expressed or are expressed at low levels in normal non-cancerous tissue. As such, neoantigens arise from one or more tumor-specific mutations. Mutation-derived neoantigens can arise from point mutations, non-synonymous mutations leading to different amino acids in the protein; read-through mutations in which a stop codon is modified or deleted, leading to translation of a longer protein with a novel tumor-specific sequence at the C-terminus; splice site mutations that lead to the inclusion of an intron in the mature mRNA and thus a unique tumor-specific protein sequence; chromosomal rearrangements that give rise to a chimeric protein with tumor-specific sequences at the junction of 2 proteins (i.e., gene fusion); frameshift mutations or deletions that lead to a new open reading frame with a novel tumor-specific protein sequence; and/or translocations. In one embodiment, the mutation is a single amino acid substitution. In other embodiments, the mutation is 2, 3, 4, 5, 6, 7, 8, 9, or 10 amino acid substitutions. In some embodiments, the length of the neoantigen peptide used to generate the neoantigen antibody is 4, 5, 6, 7, 8, 9, 10, 11, 12, 13, 14, 15, 16, 17, 18, 19, 20, 25, 30, 35, or 40 amino acids long. In one embodiment, the neoantigen peptide is 11 amino acids long. In some embodiments, the neoantigen peptide is 5-10, 5-11, 5-12, 5-13, 5-14, 5-15, 5-16, 5-17, 5-18, 5-19, 5-20, 6-10, 6-11, 6-12, 6-13, 6-14, 6-15, 6-16, 6-17, 6-18, 6-19, 6-20, 7-10, 7-11, 7-12, 7-13, 7-14, 7-15, 7-16, 7-17, 7-18, 7-19, 7-20, 8-10, 8-11, 8-12, 8-13, 8-14, 8-15, 8-16, 8-17, 8-18, 8-19, 8-20, 9-10, 9-11, 9-12, 9-13, 9-14, 9-15, 9-16, 9-17, 9-18, 9-19, 9-20, 10-15, 1-16, 10-17, 10-18, 10-19, or 10-20 amino acids in length.

Many tumor mutations are well known in the art. For example, Table 1 in the Examples section presents nine mutated epitopes from B16-F10 melanoma tumor proteins.

In some embodiments, neoantigens may be identified from tumor-secreted proteins and/or the extracellular domain of tumor membrane proteins. Immunogenicity is an important component in the selection of optimal neoantigens from which to generate antibodies. As a set of non-limiting examples, immunogenicity may be assessed by analyzing the MHC binding capacity of a neoantigen, HLA promiscuity, mutation position, predicted T cell reactivity, actual T cell reactivity, structure leading to particular conformations and resultant solvent exposure, and representation of specific amino acids. In addition, the neoantigen should have a lack of self-reactivity. Screening to determine self-reactivity can be performed to confirm that the neoantigen is restricted to tumor tissue and is not present, or is present in very low levels in the normal, non-cancerous tissue of the subject.

Some aspects of the disclosure relate to neoantigen antibodies, e.g., molecules that bind neoantigens or a fragment thereof. As used herein, the term “anti-neoantigen antibody” refers to any antibody capable of binding to a neoantigen. In some instances, the anti-neoantigen antibody can suppress the bioactivity of the neoantigen, and by extension, tumor growth.

An antibody (interchangeably used in plural form) is an immunoglobulin molecule capable of specific binding to a target, such as a carbohydrate, polynucleotide, lipid, polypeptide, etc., through at least one antigen recognition site, located in the variable region of the immunoglobulin molecule. As used herein, the term “antibody” encompasses not only intact (i.e., full-length) polyclonal or monoclonal antibodies, but also antigen-binding fragments thereof (such as Fab, Fab′, F(ab′)2, Fv), single chain (scFv), mutants thereof, fusion proteins comprising an antibody portion, humanized antibodies, chimeric antibodies, diabodies, nanobodies, linear antibodies, single chain antibodies, multi-specific antibodies (e.g., bispecific antibodies) and any other modified configuration of the immunoglobulin molecule that comprises an antigen recognition site of the required specificity, including glycosylation variants of antibodies, amino acid sequence variants of antibodies, and covalently modified antibodies. An antibody includes an antibody of any class, such as IgD, IgE, IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

A typical antibody molecule comprises a heavy chain variable region (V_{H}) and a light chain variable region (V_{L}), which are usually involved in antigen binding. The V_{H} and V_{L} regions can be further subdivided into regions of hypervariability, also known as “complementarity determining regions” (“CDR”), interspersed with regions that are more conserved, which are known as “framework regions” (“FR”). Each V_{H} and V_{L} is typically composed of three CDRs and four FRs, arranged from amino-terminus to carboxy-terminus in the following order: FR1, CDR1, FR2, CDR2, FR3, CDR3, FR4. The extent of the framework region and CDRs can be precisely identified using methodology known in the art, for example, by the Kabat definition, the Chothia definition, the IMGT definition the AbM definition, and/or the contact definition, all of which are well known in the art. See, e.g., Kabat, E. A., et al. (1991) Sequences of Proteins of Immunological Interest, Fifth Edition, U.S. Department of Health and Human Services, NIH Publication No. 91-3242, Chothia et al., (1989) Nature 342:877; Chothia, C. et al. (1987) J. Mol. Biol. 196:901-917, Al-lazikani et al (1997) J. Molec. Biol. 273:927-948; Ye et al., Nucleic Acids Res., 2013, 41:W34-40, and Almagro, J. Mol. Recognit. 17:132-143 (2004). See also hgmp.mrc.ac.uk and bioinf.org.uk/abs).

The anti-neoantigen antibodies described herein may be full-length antibodies, which contain two heavy chains and two light chains, each including a variable domain and a constant domain. Alternatively, the anti-neoantigen antibodies can be antigen-binding fragments of a full-length antibody. Examples of binding fragments encompassed within the term “antigen-binding fragment” of a full length antibody include (i) a Fab fragment, a monovalent fragment consisting of the V_{L}, V_{H}, C_{L} and C_{H}1 domains; (ii) a F(ab′)₂ fragment, a bivalent fragment including two Fab fragments linked by a disulfide bridge at the hinge region; (iii) a Fd fragment consisting of the V_{H} and C_{H}1 domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR) that retains functionality. Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv). See e.g., Bird et al. (1988) Science 242:423-426; and Huston et al. (1988) Proc. Natl. Acad. Sci. USA 85:5879-5883.

In some embodiments, the anti-neoantigen antibody as described herein can bind and inhibit the biological activity of tumor cells by at least 50% (e.g., 60%, 70%, 80%, 90%, 95% or greater). For example, the ability of the tumor cell to multiply, evade the immune system, and become invasive may be inhibited by the anti-neoantigen antibody. The apparent inhibition constant (Ki^{app} or K_{i,app}), which provides a measure of inhibitor potency, is related to the concentration of inhibitor (e.g., antibody) required to reduce enzyme activity and is not dependent on enzyme concentrations. The inhibitory activity of an anti-neoantigen antibody described herein can be determined by routine methods known in the art.

The K_{i,}^{app} value of an antibody may be determined by measuring the inhibitory effect of different concentrations of the antibody on the extent of the reaction (e.g., enzyme activity); fitting the change in pseudo-first order rate constant (v) as a function of inhibitor concentration to the modified Morrison equation (Equation 1) yields an estimate of the apparent Ki value. For a competitive inhibitor, the Ki^{app} can be obtained from the y-intercept extracted from a linear regression analysis of a plot of K_{i,}^{app} versus substrate concentration.

$$v = A \cdot \frac{( [ E ] - [ I ] - {K}_{i}^{app} ) + \sqrt{{( [ E ] - [ I ] - {K}_{i}^{app} )}^{2} + 4 [ E ]} \cdot {K}_{i}^{app}}{2} \qquad ( Equation 1 )$$

Where A is equivalent to vₒ/E, the initial velocity (vₒ) of the enzymatic reaction in the absence of inhibitor (I) divided by the total enzyme concentration (E).

In some embodiments, the anti-neoantigen antibody described herein may have a Ki^{app} value of 1000, 900, 800, 700, 600, 500, 400, 300, 200, 100, 50, 40, 30, 20, 19, 18, 17, 16, 15, 14, 13, 12, 11, 10, 9, 8, 7, 6, 5 pM or less for the target neoantigen or neoantigen epitope. In some embodiments, the anti-neoantigen antibody may have a lower Ki^{app} for a first target (e.g., one epitope of a neoantigen) relative to a second target (e.g., a second epitope of the neoantigen). Differences in Ki^{app} (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some examples, the anti-neoantigen antibody inhibits a first antigen (e.g., a first protein in a first conformation or mimic thereof) better relative to a second antigen (e.g., the same first protein in a second conformation or mimic thereof; or a second protein). In some embodiments, any of the anti-neoantigen antibodies may be further affinity matured to reduce the Ki^{app} of the antibody to the target neoantigen or neoantigenic epitope thereof.

The antibodies described herein can be murine, rat, rabbit, human, or any other origin (including chimeric or humanized antibodies). Such antibodies are non-naturally occurring, i.e., would not be produced in an animal without human act (e.g., immunizing such an animal with a desired antigen or fragment thereof or isolated from antibody libraries). In some instances, the antibodies are pooled antibodies (e.g., from more than one source). In other instances, the antibodies are from one donor. In one instance, the anti-neoantigen antibodies are pooled human antibodies.

Any of the antibodies described herein can be either monoclonal or polyclonal. A “monoclonal antibody” refers to a homogenous antibody population and a “polyclonal antibody” refers to a heterogeneous antibody population. These two terms do not limit the source of an antibody or the manner in which it is made.

In one example, the antibody used in the methods described herein is a humanized antibody. Humanized antibodies refer to forms of non-human (e.g., murine) antibodies that are specific chimeric immunoglobulins, immunoglobulin chains, or antigen-binding fragments thereof that contain minimal sequence derived from non-human immunoglobulin. For the most part, humanized antibodies are human immunoglobulins (recipient antibody) in which residues from a CDR of the recipient are replaced by residues from a CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported CDR or framework sequences, but are included to further refine and optimize antibody performance. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fe), typically that of a human immunoglobulin. Antibodies may have Fc regions modified as described in WO 99/58572. Other forms of humanized antibodies have one or more CDRs (one, two, three, four, five, or six) which are altered with respect to the original antibody, which are also termed one or more CDRs “derived from” one or more CDRs from the original antibody. Humanized antibodies may also involve affinity maturation.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions can be used to substitute for the corresponding residues in the human acceptor genes.

In another example, the antibody described herein is a chimeric antibody, which can include a heavy constant region and a light constant region from a human antibody. Chimeric antibodies refer to antibodies having a variable region or part of variable region from a first species and a constant region from a second species. Typically, in these chimeric antibodies, the variable region of both light and heavy chains mimics the variable regions of antibodies derived from one species of mammals (e.g., a non-human mammal such as mouse, rabbit, and rat), while the constant portions are homologous to the sequences in antibodies derived from another mammal such as human. In some embodiments, amino acid modifications can be made in the variable region and/or the constant region. Modifications can include naturally occurring amino acids and non-naturally occurring amino acids. Examples of non-naturally occurring amino acids are modifications that are not isotypic and can be found in U.S. Pat. No. 6,586,207; WO 98/48032; WO 03/073238; US2004-0214988A1; WO 05/35727A2; WO 05/74524A2; J. W. Chin et al., (2002), Journal of the American Chemical Society 124:9026-9027; J. W. Chin, & P. G. Schultz, (2002), ChemBioChem 11:1135-1137; J. W. Chin, et al., (2002), PICAS United States of America 99:11020-11024; and, L. Wang, & P. G. Schultz, (2002), Chem. 1-10, each of which is incorporated by reference herein in its entirety.

In some embodiments, the anti-neoantigen antibodies described herein specifically bind to the corresponding target neoantigen or an epitope thereof. An antibody that “specifically binds” to a neoantigen or an epitope is a term well understood in the art. A molecule is said to exhibit “specific binding” if it reacts more frequently, more rapidly, with greater duration and/or with greater affinity with a particular target antigen than it does with alternative targets. An antibody “specifically binds” to a target antigen or epitope if it binds with greater affinity, avidity, more readily, and/or with greater duration than it binds to other substances. For example, an antibody that specifically (or preferentially) binds to a neoantigen or fragment thereof (e.g., epitope) is an antibody that binds this target antigen with greater affinity, avidity, more readily, and/or with greater duration than it binds to other neoantigens or other epitopes in the same neoantigen. It is also understood with this definition that, for example, an antibody that specifically binds to a first target neoantigen may or may not specifically or preferentially bind to a second target neoantigen. As such, “specific binding” or “preferential binding” does not necessarily require (although it can include) exclusive binding. In some examples, an antibody that “specifically binds” to a target antigen or an epitope thereof may not bind to other antigens or other epitopes in the same antigen (i.e., only baseline binding activity can be detected in a conventional method). In some embodiments, the antibodies described herein specifically bind to a selected epitope of a neoantigen.

In some embodiments, an anti-neoantigen antibody as described herein has a suitable binding affinity for the target antigen (e.g., neoantigen) or epitope(s) thereof. As used herein, “binding affinity” refers to the apparent association constant or K_{A}. The K_{A} is the reciprocal of the dissociation constant (K_{D}). The anti-neoantigen antibodies described herein may have a binding affinity (K_{D}) of at least 10⁻⁵, 10⁻⁶, 10⁻⁷, 10⁻⁸, 10⁻⁹, 10⁻¹⁰ M, or lower for the target neoantigen or epitope thereof. An increased binding affinity corresponds to a decreased K_{D}. Higher affinity binding of an antibody for a first antigen relative to a second antigen can be indicated by a higher K_{A} (or a smaller numerical value K_{D}) for binding the first antigen than the K_{A} (or numerical value K_{D}) for binding the second antigen. In such cases, the antibody has specificity for the first antigen (e.g., a first protein in a first conformation or mimic thereof) relative to the second antigen (e.g., the same first protein in a second conformation or mimic thereof; or a second protein). In some embodiments, the anti-neoantigen antibodies described herein have a higher binding affinity (a higher K_{A} or smaller K_{D}) to a specific neoantigen as compared to the binding affinity to a second neoantigen. Differences in binding affinity (e.g., for specificity or other comparisons) can be at least 1.5, 2, 3, 4, 5, 10, 15, 20, 37.5, 50, 70, 80, 91, 100, 500, 1000, 10,000 or 10⁵ fold. In some embodiments, any of the anti-neoantigen antibodies may be further affinity matured to increase the binding affinity of the antibody to the target neoantigen or antigenic epitope thereof.

Binding affinity (or binding specificity) can be determined by a variety of methods including equilibrium dialysis, equilibrium binding, gel filtration, ELISA, surface plasmon resonance, or spectroscopy (e.g., using a fluorescence assay). Exemplary conditions for evaluating binding affinity are in HBS-P buffer (10 mM HEPES pH7.4, 150 mM NaCl, 0.005% (v/v) Surfactant P20). These techniques can be used to measure the concentration of bound binding protein as a function of target protein concentration. The concentration of bound binding protein ([Bound]) is generally related to the concentration of free target protein ([Free]) by the following equation:

$$[ Bound ] = [ Free ] / ( Kd + [ Free ] )$$

It is not always necessary to make an exact determination of K_{A}, though, since sometimes it is sufficient to obtain a quantitative measurement of affinity, e.g., determined using a method such as ELISA or FACS analysis, is proportional to K_{A}, and thus can be used for comparisons, such as determining whether a higher affinity is, e.g., 2-fold higher, to obtain a qualitative measurement of affinity, or to obtain an inference of affinity, e.g., by activity in a functional assay, e.g., an in vitro or in vivo assay.

In some embodiments, the anti-neoantigen antibodies described herein bind to the same epitope as any of the exemplary antibodies described herein or competes against the exemplary antibody from binding to the neoantigen. An “epitope” refers to the site on a target neoantigen that is recognized and bound by an antibody. The site can be entirely composed of amino acid components, entirely composed of chemical modifications of amino acids of the protein (e.g., glycosyl moieties), or composed of combinations thereof. Overlapping epitopes include at least one common amino acid residue. An epitope can be linear, which is typically 6-15 amino acids in length. Alternatively, the epitope can be conformational. The epitope to which an antibody binds can be determined by routine technology, for example, the epitope mapping method (see, e.g., descriptions below). An antibody that binds the same epitope as an exemplary antibody described herein may bind to exactly the same epitope or a substantially overlapping epitope (e.g., containing less than 3 non-overlapping amino acid residue, less than 2 non-overlapping amino acid residues, or only 1 non-overlapping amino acid residue) as the exemplary antibody. Whether two antibodies compete against each other from binding to the cognate antigen can be determined by a competition assay, which is well known in the art.

Also within the scope of the present disclosure are functional variants of any of the exemplary anti-neoantigen antibodies as disclosed herein. Such functional variants are substantially similar to the exemplary antibody, both structurally and functionally. A functional variant comprises substantially the same V_{H} and V_{L} CDRs as the exemplary antibody. For example, it may comprise only up to 5 (e.g., 4, 3, 2, or 1) amino acid residue variations in the total CDR regions of the antibody and binds the same epitope of the neoantigen with substantially similar affinity (e.g., having a K_{D} value in the same order). Alternatively or in addition, the amino acid residue variations are conservative amino acid residue substitutions. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references which compile such methods, e.g. Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F. M. Ausubel, et al., eds., John Wiley & Sons, Inc., New York. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D.

In some embodiments, the anti-neoantigen antibody may comprise heavy chain CDRs that share at least 80% (e.g., 85%, 90%, 95%, or 98%) sequence identity, individually or collectively, with the V_{H} CDRs of an antibody described herein. Alternatively or in addition, the anti-neoantigen antibody may comprise light chain CDRs that share at least 80% (e.g., 85%, 90%, 95%, or 98%) sequence identity, individually or collectively, with the V_{L} CDRs as an antibody described herein.

The “percent identity” of two amino acid sequences is determined using the algorithm of Karlin and Altschul Proc. Natl. Acad. Sci. USA 87:2264-68, 1990, modified as in Karlin and Altschul Proc. Natl. Acad. Sci. USA 90:5873-77, 1993. Such an algorithm is incorporated into the NBLAST and XBLAST programs (version 2.0) of Altschul, et al. J. Mol. Biol. 215:403-10, 1990. BLAST protein searches can be performed with the XBLAST program, score=50, wordlength=3 to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used.

In some embodiments, the heavy chain of any of the anti-neoantigen antibodies as described herein may further comprise a heavy chain constant region (CH) or a portion thereof (e.g., CH1, CH2, CH3, or a combination thereof). The heavy chain constant region can of any suitable origin, e.g., human, mouse, rat, or rabbit. In one specific example, the heavy chain constant region is from a human IgG (a gamma heavy chain) of any IgG subfamily as described herein. In one example, the constant region is from human IgG4, an exemplary amino acid sequence of which is provided below (SEQ ID NO: 19):

ASTKGPSVFP LAPCSRSTSE STAALGCLVK
DYFPEPVTVS WNSGALTSGV HTFPAVLQSS
GLYSLSSVVT VPSSSLGTKT YTCNVDHKPS
NTKVDKRVES KYGPPCPSCP APEFLGGPSV
FLFPPKPKDT LMISRTPEVT CVVVDVSQED
PEVQFNWYVD GVEVHNAKTK PREEQFNSTY
RVVSVLTVLH QDWLNGKEYK CKVSNKGLPS
SIEKTISKAK GQPREPQVYT LPPSQEEMTK
NQVSLTCLVK GFYPSDIAVE WESNGQPENN
YKTTPPVLDS DGSFFLYSRL TVDKSRWQEG
NVFSCSVMHE ALHNHYTQKS LSLSLGK

In some embodiments, the anti-neoantigen antibody comprises the heavy chain constant region of SEQ ID NO: 19, or a variant thereof, which may contain an S/P substitution at the position as indicated (boldfaced and underlined). Alternatively, the heavy chain constant region of the antibodies described herein may comprise a single domain (e.g., CH1, CH2, or CH3) or a combination of any of the single domains, of a constant region (e.g., SEQ ID NO: 19).

When needed, the anti-neoantigen antibody as described herein may comprise a modified constant region. For example, it may comprise a modified constant region that is immunologically inert, e.g., does not trigger complement mediated lysis, or does not stimulate antibody-dependent cell mediated cytotoxicity (ADCC). ADCC activity can be assessed using methods disclosed in U.S. Pat. No. 5,500,362. In other embodiments, the constant region is modified as described in Eur. J. Immunol. (1999) 29:2613-2624; PCT Application No. PCT/GB99/01441; and/or UK Patent Application No. 9809951.8.

Any of the anti-neoantigen antibodies described herein may comprise a light chain that further comprises a light chain constant region, which can be any CL known in the art. In some examples, the CL is a kappa light chain. In other examples, the CL is a lambda light chain.

Antibody heavy and light chain constant regions are well known in the art, e.g., those provided in the IMGT database (www.imgt.org) or at www.vbase2.org/vbstat.php., both of which are incorporated by reference herein.

Antibodies capable of binding a neoantigen or epitope thereof as described herein can be made by any method known in the art. See, for example, Harlow and Lane, (1998) Antibodies: A Laboratory Manual, Cold Spring Harbor Laboratory, New York.

In some embodiments, antibodies specific to a target neoantigen can be made by the conventional hybridoma technology. The full-length target antigen or a fragment thereof, optionally coupled to a carrier protein such as KLH, can be used to immunize a host animal for generating antibodies binding to that antigen. The route and schedule of immunization of the host animal are generally in keeping with established and conventional techniques for antibody stimulation and production, as further described herein. General techniques for production of mouse, humanized, and human antibodies are known in the art and are described herein. It is contemplated that any mammalian subject including humans or antibody producing cells therefrom can be manipulated to serve as the basis for production of mammalian, including human hybridoma cell lines. Typically, the host animal is inoculated intraperitoneally, intramuscularly, orally, subcutaneously, intraplantar, and/or intradermally with an amount of immunogen, including as described herein.

Hybridomas can be prepared from the lymphocytes and immortalized myeloma cells using the general somatic cell hybridization technique of Kohler, B. and Milstein, C. (1975) Nature 256:495-497 or as modified by Buck, D. W., et al., In Vitro, 18:377-381 (1982). Available myeloma lines, including but not limited to X63-Ag8.653 and those from the Salk Institute, Cell Distribution Center, San Diego, Calif., USA, may be used in the hybridization. Generally, the technique involves fusing myeloma cells and lymphoid cells using a fusogen such as polyethylene glycol, or by electrical means well known to those skilled in the art. After the fusion, the cells are separated from the fusion medium and grown in a selective growth medium, such as hypoxanthine-aminopterin-thymidine (HAT) medium, to eliminate unhybridized parent cells. Any of the media described herein, supplemented with or without serum, can be used for culturing hybridomas that secrete monoclonal antibodies. As another alternative to the cell fusion technique, EBV immortalized B cells may be used to produce the anti-neoantigen monoclonal antibodies described herein. The hybridomas are expanded and subcloned, if desired, and supernatants are assayed for anti-immunogen activity by conventional immunoassay procedures (e.g., radioimmunoassay, enzyme immunoassay, or fluorescence immunoassay).

Hybridomas that may be used as source of antibodies encompass all derivatives, progeny cells of the parent hybridomas that produce monoclonal antibodies capable of interfering with the ZIKV bioactivity. Hybridomas that produce such antibodies may be grown in vitro or in vivo using known procedures. The monoclonal antibodies may be isolated from the culture media or body fluids, by conventional immunoglobulin purification procedures such as ammonium sulfate precipitation, gel electrophoresis, dialysis, chromatography, and ultrafiltration, if desired. Undesired activity if present, can be removed, for example, by running the preparation over adsorbents made of the immunogen attached to a solid phase and eluting or releasing the desired antibodies off the immunogen. Immunization of a host animal with a target antigen or a fragment containing the target amino acid sequence conjugated to a protein that is immunogenic in the species to be immunized, e.g., keyhole limpet hemocyanin, serum albumin, bovine thyroglobulin, or soybean trypsin inhibitor using a bifunctional or derivatizing agent, for example maleimidobenzoyl sulfosuccinimide ester (conjugation through cysteine residues), N-hydroxysuccinimide (through lysine residues), glutaraldehyde, succinic anhydride, SOCl, or R1N═C═NR, where R and R1 are different alkyl groups, can yield a population of antibodies (e.g., monoclonal antibodies).

If desired, an antibody (monoclonal or polyclonal) of interest (e.g., produced by a hybridoma) may be sequenced and the polynucleotide sequence may then be cloned into a vector for expression or propagation. The sequence encoding the antibody of interest may be maintained in vector in a host cell and the host cell can then be expanded and frozen for future use. In an alternative, the polynucleotide sequence may be used for genetic manipulation to “humanize” the antibody or to improve the affinity (affinity maturation), or other characteristics of the antibody. For example, the constant region may be engineered to more resemble human constant regions to avoid immune response if the antibody is used in clinical trials and treatments in humans. It may be desirable to genetically manipulate the antibody sequence to obtain greater affinity to the target antigen and greater efficacy in inhibiting the bioactivity of a tumor (e.g., tumor cells). It will be apparent to one of skill in the art that one or more polynucleotide changes can be made to the antibody and still maintain its binding specificity to the target neoantigen.

In other embodiments, fully human antibodies can be obtained by using commercially available mice that have been engineered to express specific human immunoglobulin proteins. Transgenic animals that are designed to produce a more desirable (e.g., fully human antibodies) or more robust immune response may also be used for generation of humanized or human antibodies. Examples of such technology are Xenomouse® from Amgen, Inc. (Fremont, Calif.) and HuMAb-Mouse® and TC Mouse™ from Medarex, Inc. (Princeton, N.J.). In another alternative, antibodies may be made recombinantly by phage display or yeast technology. See, for example, U.S. Pat. Nos. 5,565,332; 5,580,717; 5,733,743; and 6,265,150; and Winter et al., (1994) Annu. Rev. Immunol. 12:433-455. Recombinant mammalian expression systems or recombinant bacterial expression systems may also be used to generate the anti-neoantigen antibodies of the instant disclosure. Alternatively, the phage display technology (McCafferty et al., (1990) Nature 348:552-553) can be used to produce human antibodies and antibody fragments in vitro, from immunoglobulin variable (V) domain gene repertoires from unimmunized donors.

Alternatively, antibodies capable of binding to the target antigens as described herein may be isolated from a suitable antibody library via routine practice. Antibody libraries, which contain a plurality of antibody components, can be used to identify antibodies that bind to a specific target neoantigen following routine selection processes as known in the art. In the selection process, an antibody library can be probed with the target antigen or a fragment thereof and members of the library that are capable of binding to the target antigen can be isolated, typically by retention on a support. Such screening process may be performed by multiple rounds (e.g., including both positive and negative selections) to enrich the pool of antibodies capable of binding to the target antigen. Individual clones of the enriched pool can then be isolated and further characterized to identify those having desired binding activity and biological activity. Sequences of the heavy chain and light chain variable domains can also be determined via conventional methodology.

There are a number of routine methods known in the art to identify and isolate antibodies capable of binding to the target neoantigens described herein, including phage display, yeast display, ribosomal display, or mammalian display technology.

As an example, phage displays typically use a covalent linkage to bind the protein (e.g., antibody) component to a bacteriophage coat protein. The linkage results from translation of a nucleic acid encoding the antibody component fused to the coat protein. The linkage can include a flexible peptide linker, a protease site, or an amino acid incorporated as a result of suppression of a stop codon. Phage display is described, for example, in U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8 and Hoet et al. (2005) Nat Biotechnol. 23(3)344-8. Bacteriophage displaying the protein component can be grown and harvested using standard phage preparatory methods, e.g. PEG precipitation from growth media. After selection of individual display phages, the nucleic acid encoding the selected protein components can be isolated from cells infected with the selected phages or from the phage themselves, after amplification. Individual colonies or plaques can be selected, and then the nucleic acid may be isolated and sequenced.

Other display formats include cell-based display (see, e.g., WO 03/029456), protein-nucleic acid fusions (see, e.g., U.S. Pat. No. 6,207,446), ribosome display (See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30; and Schaffitzel et al. (1999) J Immunol Methods. 231(1-2):119-35), and E. coli periplasmic display (J Immunol Methods. 2005 Nov. 22; PMID: 16337958).

After display library members are isolated for binding to the target antigen, each isolated library member can be also tested for its ability to bind to a non-target molecule to evaluate its binding specificity. Examples of non-target molecules include streptavidin on magnetic beads, blocking agents such as bovine serum albumin, non-fat bovine milk, soy protein, any capturing or target immobilizing monoclonal antibody, or non-transfected cells which do not express the target. A high-throughput ELISA screen can be used to obtain the data, for example. The ELISA screen can also be used to obtain quantitative data for binding of each library member to the target as well as for cross species reactivity to related targets or subunits of the target antigen and also under different condition such as pH 6 or pH 7.5. The non-target and target binding data are compared (e.g., using a computer and software) to identify library members that specifically bind to the target.

After selecting candidate library members that bind to a target, each candidate library member can be further analyzed, e.g., to further characterize its binding properties for the target, e.g., a specific neoantigen. Each candidate library member can be subjected to one or more secondary screening assays. The assay can be for a binding property, a catalytic property, an inhibitory property, a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use a display library member directly, a recombinant polypeptide produced from the nucleic acid encoding the selected polypeptide, or a synthetic peptide synthesized based on the sequence of the selected polypeptide. In the case of selected Fabs, the Fabs can be evaluated or can be modified and produced as intact IgG proteins. Exemplary assays for binding properties are described below.

Binding proteins can also be evaluated using an ELISA assay. For example, each protein is contacted to a microtiter plate whose bottom surface has been coated with the target, e.g., a limiting amount of the target. The plate is washed with buffer to remove non-specifically bound polypeptides. Then the amount of the binding protein bound to the target on the plate is determined by probing the plate with an antibody that can recognize the binding protein, e.g., a tag or constant portion of the binding protein. The antibody is linked to a detection system (e.g., an enzyme such as alkaline phosphatase or horseradish peroxidase (HRP) which produces a colorimetric product when appropriate substrates are provided, chemiluminescent substrates, or fluorescent substrates).

Alternatively, the ability of a binding protein described herein to bind a target antigen can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means, e.g., using a fluorimeter. By titrating the amount of the first or second binding molecule, a binding curve can be generated to estimate the equilibrium binding constant.

Surface plasmon resonance (SPR) can be used to analyze the interaction of a binding protein and a target antigen. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of SPR). The changes in the refractivity generate a detectable signal, which are measured as an indication of real-time reactions between biological molecules. Methods for using SPR are described, for example, in U.S. Pat. No. 5,641,640; Raether, 1988, Surface Plasmons Springer Verlag; Sjolander and Urbaniczky, 1991, Anal. Chem. 63:2338-2345; Szabo et al., 1995, Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{D}), and kinetic parameters, including Kₒₙ and K_{off}, for the binding of a binding protein to a target. Such data can be used to compare different biomolecules. For example, selected proteins from an expression library can be compared to identify proteins that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by x-ray crystallography or NMR). As a result, an understanding of the physical interaction between the protein and its target can be formulated and used to guide other design processes.

As a further example, cellular assays may be used. Binding proteins can be screened for ability to bind to cells which transiently or stably express and display the target of interest on the cell surface. For example, a target neoantigen's binding proteins can be fluorescently labeled and binding to the neoantigen in the presence or absence of antagonistic antibody can be detected by a change in fluorescence intensity using flow cytometry e.g., a FACS machine.

Antigen-binding fragments of an intact antibody (full-length antibody) can be prepared via routine methods. For example, F(ab′)2 fragments can be produced by pepsin digestion of an antibody molecule, and Fab fragments that can be generated by reducing the disulfide bridges of F(ab′)2 fragments.

Genetically engineered antibodies, such as humanized antibodies, chimeric antibodies, single-chain antibodies, and bi-specific antibodies, can be produced via, e.g., conventional recombinant technology. In one example, DNA encoding a monoclonal antibodies specific to a target antigen can be readily isolated and sequenced using conventional procedures (e.g., by using oligonucleotide probes that are capable of binding specifically to genes encoding the heavy and light chains of the monoclonal antibodies). Once isolated, the DNA may be placed into one or more expression vectors, which are then transfected into host cells such as HEK293 cells, E. coli cells, simian COS cells, Chinese hamster ovary (CHO) cells, or myeloma cells that do not otherwise produce immunoglobulin protein, to obtain the synthesis of monoclonal antibodies in the recombinant host cells. See, e.g., PCT Publication No. WO 87/04462. The DNA can then be modified, for example, by substituting the coding sequence for human heavy and light chain constant domains in place of the homologous murine sequences, Morrison et al., (1984) Proc. Nat. Acad. Sci. 81:6851, or by covalently joining to the immunoglobulin coding sequence all or part of the coding sequence for a non-immunoglobulin polypeptide. In that manner, genetically engineered antibodies, such as “chimeric” or “hybrid” antibodies; can be prepared that have the binding specificity of a target antigen.

Techniques developed for the production of “chimeric antibodies” are well known in the art. See, e.g., Morrison et al. (1984) Proc. Natl. Acad. Sci. USA 81, 6851; Neuberger et al. (1984) Nature 312, 604; and Takeda et al. (1984) Nature 314:452.

Methods for constructing humanized antibodies are also well known in the art. See, e.g., Queen et al., Proc. Natl. Acad. Sci. USA, 86:10029-10033 (1989). In one example, variable regions of V_{H} and V_{L} of a parent non-human antibody are subjected to three-dimensional molecular modeling analysis following methods known in the art. Next, framework amino acid residues predicted to be important for the formation of the correct CDR structures are identified using the same molecular modeling analysis. In parallel, human V_{H} and V_{L} chains having amino acid sequences that are homologous to those of the parent non-human antibody are identified from any antibody gene database using the parent V_{H} and V_{L} sequences as search queries. Human V_{H} and V_{L} acceptor genes are then selected.

The CDR regions within the selected human acceptor genes can be replaced with the CDR regions from the parent non-human antibody or functional variants thereof. When necessary, residues within the framework regions of the parent chain that are predicted to be important in interacting with the CDR regions (see above description) can be used to substitute for the corresponding residues in the human acceptor genes.

A single-chain antibody can be prepared via recombinant technology by linking a nucleotide sequence coding for a heavy chain variable region and a nucleotide sequence coding for a light chain variable region. Preferably, a flexible linker is incorporated between the two variable regions. Alternatively, techniques described for the production of single chain antibodies (U.S. Pat. Nos. 4,946,778 and 4,704,692) can be adapted to produce a phage or yeast scFv library and scFv clones specific to a target neoantigen can be identified from the library following routine procedures. Positive clones can be subjected to further screening to identify those that inhibit tumor (e.g., tumor cell) bioactivity.

Antibodies obtained following a method known in the art and described herein can be characterized using methods well known in the art. For example, one method is to identify the epitope to which the antigen binds, or “epitope mapping.” There are many methods known in the art for mapping and characterizing the location of epitopes on proteins, including solving the crystal structure of an antibody-antigen complex, competition assays, gene fragment expression assays, and synthetic peptide-based assays, as described, for example, in Chapter 11 of Harlow and Lane, Using Antibodies, a Laboratory Manual, Cold Spring Harbor Laboratory Press, Cold Spring Harbor, N.Y., 1999. In an additional example, epitope mapping can be used to determine the sequence, to which an antibody binds. The epitope can be a linear epitope, i.e., contained in a single stretch of amino acids, or a conformational epitope formed by a three-dimensional interaction of amino acids that may not necessarily be contained in a single stretch (primary structure linear sequence). Peptides of varying lengths (e.g., at least 4-6 amino acids long, in some embodiments, 11 amino acids long) can be isolated or synthesized (e.g., recombinantly) and used for binding assays with an antibody. In another example, the epitope to which the antibody binds can be determined in a systematic screening by using overlapping peptides derived from the target antigen sequence and determining binding by the antibody. According to the gene fragment expression assays, the open reading frame encoding the target neoantigen is fragmented either randomly or by specific genetic constructions and the reactivity of the expressed fragments of the antigen with the antibody to be tested is determined. The gene fragments may, for example, be produced by PCR and then transcribed and translated into protein in vitro, in the presence of radioactive amino acids. The binding of the antibody to the radioactively labeled neoantigen fragments is then determined by immunoprecipitation and gel electrophoresis. Certain epitopes can also be identified by using large libraries of random peptide sequences displayed on the surface of phage particles (phage libraries). Alternatively, a defined library of overlapping peptide fragments can be tested for binding to the test antibody in simple binding assays. In an additional example, mutagenesis of an antigen binding domain, domain swapping experiments and alanine scanning mutagenesis can be performed to identify residues required, sufficient, and/or necessary for epitope binding. For example, domain swapping experiments can be performed using a mutant of a target antigen in which various fragments of the neoantigen polypeptide have been replaced (swapped) with sequences from a closely related, but antigenically distinct protein. By assessing binding of the antibody to the mutant neoantigen, the importance of the particular antigen fragment to antibody binding can be assessed.

Alternatively, competition assays can be performed using other antibodies known to bind to the same antigen to determine whether an antibody binds to the same epitope as the other antibodies. Competition assays are well known to those of skill in the art.

In some examples, an anti-neoantigen antibody is prepared by recombinant technology as exemplified below.

Nucleic acids encoding the heavy and light chain of an anti-neoantigen antibody as described herein can be cloned into one expression vector, each nucleotide sequence being in operable linkage to a suitable promoter. In one example, each of the nucleotide sequences encoding the heavy chain and light chain is in operable linkage to a distinct prompter. Alternatively, the nucleotide sequences encoding the heavy chain and the light chain can be in operable linkage with a single promoter, such that both heavy and light chains are expressed from the same promoter. When necessary, an internal ribosomal entry site (TRES) can be inserted between the heavy chain and light chain encoding sequences.

In some examples, the nucleotide sequences encoding the two chains of the antibody are cloned into two vectors, which can be introduced into the same or different cells. When the two chains are expressed in different cells, each of them can be isolated from the host cells expressing such and the isolated heavy chains and light chains can be mixed and incubated under suitable conditions allowing for the formation of the antibody.

Generally, a nucleic acid sequence encoding one or all chains of an antibody can be cloned into a suitable expression vector in operable linkage with a suitable promoter using methods known in the art. For example, the nucleotide sequence and vector can be contacted, under suitable conditions, with a restriction enzyme to create complementary ends on each molecule that can pair with each other and be joined together with a ligase. Alternatively, synthetic nucleic acid linkers can be ligated to the termini of a gene. These synthetic linkers contain nucleic acid sequences that correspond to a particular restriction site in the vector. The selection of expression vectors/promoter would depend on the type of host cells for use in producing the antibodies.

A variety of promoters can be used for expression of the antibodies described herein, including, but not limited to, cytomegalovirus (CMV) intermediate early promoter, a viral LTR such as the Rous sarcoma virus LTR, HIV-LTR, HTLV-1 LTR, the simian virus 40 (SV40) early promoter, E. coli lac UV5 promoter, and the herpes simplex tk virus promoter.

Regulatable promoters can also be used. Such regulatable promoters include those using the lac repressor from E. coli as a transcription modulator to regulate transcription from lac operator-bearing mammalian cell promoters [Brown, M. et al., Cell, 49:603-612 (1987)], those using the tetracycline repressor (tetR) [Gossen, M., and Bujard, H., Proc. Natl. Acad. Sci. USA 89:5547-5551 (1992); Yao, F. et al., Human Gene Therapy, 9:1939-1950 (1998); Shockelt, P., et al., Proc. Natl. Acad. Sci. USA, 92:6522-6526 (1995)]. Other systems include FK506 dimer, VP16 or p65 using astradiol, RU486, diphenol murislerone, or rapamycin. Inducible systems are available from Invitrogen, Clontech and Ariad.

Regulatable promoters that include a repressor with the operon can be used. In one embodiment, the lac repressor from E. coli can function as a transcriptional modulator to regulate transcription from lac operator-bearing mammalian cell promoters [M. Brown et al., Cell, 49:603-612 (1987)]; Gossen and Bujard (1992); [M. Gossen et al., Natl. Acad. Sci. USA, 89:5547-5551 (1992)] combined the tetracycline repressor (tetR) with the transcription activator (VP 16) to create a tetR-mammalian cell transcription activator fusion protein, tTa (tetR-VP 16), with the tetO-bearing minimal promoter derived from the human cytomegalovirus (hCMV) major immediate-early promoter to create a tetR-tet operator system to control gene expression in mammalian cells. In one embodiment, a tetracycline inducible switch is used. The tetracycline repressor (tetR) alone, rather than the tetR-mammalian cell transcription factor fusion derivatives can function as potent trans-modulator to regulate gene expression in mammalian cells when the tetracycline operator is properly positioned downstream for the TATA element of the CMVIE promoter (Yao et al., Human Gene Therapy, 10(16):1392-1399 (2003)). One particular advantage of this tetracycline inducible switch is that it does not require the use of a tetracycline repressor-mammalian cells transactivator or repressor fusion protein, which in some instances can be toxic to cells (Gossen et al., Natl. Acad. Sci. USA, 89:5547-5551 (1992); Shockett et al., Proc. Natl. Acad. Sci. USA, 92:6522-6526 (1995)), to achieve its regulatable effects.

Additionally, the vector can contain, for example, some or all of the following: a selectable marker gene, such as the neomycin gene for selection of stable or transient transfectants in mammalian cells; enhancer/promoter sequences from the immediate early gene of human CMV for high levels of transcription; transcription termination and RNA processing signals from SV40 for mRNA stability; SV40 polyoma origins of replication and ColE1 for proper episomal replication; internal ribosome binding sites (IRESes), versatile multiple cloning sites; and T7 and SP6 RNA promoters for in vitro transcription of sense and antisense RNA. Suitable vectors and methods for producing vectors containing transgenes are well known and available in the art.

Examples of polyadenylation signals useful to practice the methods described herein include, but are not limited to, human collagen I polyadenylation signal, human collagen II polyadenylation signal, and SV40 polyadenylation signal.

One or more vectors (e.g., expression vectors) comprising nucleic acids encoding any of the antibodies may be introduced into suitable host cells for producing the antibodies. The host cells can be cultured under suitable conditions for expression of the antibody or any polypeptide chain thereof. Such antibodies or polypeptide chains thereof can be recovered by the cultured cells (e.g., from the cells or the culture supernatant) via a conventional method, e.g., affinity purification. If necessary, polypeptide chains of the antibody can be incubated under suitable conditions for a suitable period of time allowing for production of the antibody.

In some embodiments, methods for preparing an antibody described herein involve a recombinant expression vector that encodes both the heavy chain and the light chain of an anti-neoantigen antibody, as also described herein. The recombinant expression vector can be introduced into a suitable host cell (e.g., a dhfr− CHO cell) by a conventional method, e.g., calcium phosphate-mediated transfection. Positive transformant host cells can be selected and cultured under suitable conditions allowing for the expression of the two polypeptide chains that form the antibody, which can be recovered from the cells or from the culture medium. When necessary, the two chains recovered from the host cells can be incubated under suitable conditions allowing for the formation of the antibody.

In one example, two recombinant expression vectors are provided, one encoding the heavy chain of the anti-neoantigen antibody and the other encoding the light chain of the anti-neoantigen antibody. Both of the two recombinant expression vectors can be introduced into a suitable host cell (e.g., dhfr− CHO cell) by a conventional method, e.g., calcium phosphate-mediated transfection. Alternatively, each of the expression vectors can be introduced into a suitable host cells. Positive transformants can be selected and cultured under suitable conditions allowing for the expression of the polypeptide chains of the antibody. When the two expression vectors are introduced into the same host cells, the antibody produced therein can be recovered from the host cells or from the culture medium. If necessary, the polypeptide chains can be recovered from the host cells or from the culture medium and then incubated under suitable conditions allowing for formation of the antibody. When the two expression vectors are introduced into different host cells, each of them can be recovered from the corresponding host cells or from the corresponding culture media. The two polypeptide chains can then be incubated under suitable conditions for formation of the antibody.

Standard molecular biology techniques are used to prepare the recombinant expression vector, transfect the host cells, select for transformants, culture the host cells and recovery of the antibodies from the culture medium. For example, some antibodies can be isolated by affinity chromatography with a Protein A or Protein G coupled matrix.

Any of the nucleic acids encoding the heavy chain, the light chain, or both of an anti-neoantigen antibody as described herein, vectors (e.g., expression vectors) containing such, and host cells comprising the vectors are within the scope of the present disclosure.

Anti-neoantigen antibodies thus prepared can be characterized using methods known in the art, whereby reduction, amelioration, or neutralization of tumor (e.g., tumor cell) biological activity is detected and/or measured. For example, an ELISA-type assay may be suitable for qualitative or quantitative measurement of tumor (e.g., tumor cell) bioactivity neutralization.

The present disclosure provides pharmaceutical compositions comprising two or more anti-neoantigen antibodies described herein and uses of such for neutralizing tumor (e.g., tumor cell) bioactivity. For example, the antibodies and antigen-binding antibody fragments thereof described herein may be used to treat cancer in a subject. As the antibodies bind neoantigens with high specificity, they may be used to treat a subject having cancer.

As described herein, effector cells may be coated with the anti-neoantigen antibodies ex vivo, which reduces the amount of antibody required to cause tumor inhibition. Previous studies have demonstrated multiple types of effector cells derived from spleen, peripheral blood, marrow, and peritoneum can mediate cytotoxicity. For example, serum from Guinea pigs immunized with chicken red blood cells was shown to bind firmly to monocyte-macrophages, non-phagocytic lymphocytes, and neutrophils. Binding of the serum rendered the different effector cells cytotoxic to chicken red blood cells²⁴. Effector cells derived from human blood demonstrated a range of cytotoxic activity when armed with rabbit antibodies. Non-lymphocytes increased the initial rate of cytotoxicity but purified lymphocytes appeared to have more complete cytotoxicity over time that polymorphonuclear leukocytes and monocytes²⁵. Neutrophils, directed by antibodies, have been shown to attack target cells by a unique method of trogocytosis²⁶. Using only one cell type as is done with T cell therapy, does not take full advantage of multiple mechanisms of tumor cell destruction by different effector cells.

Despite the fact that multiple effector cell types can participate in antibody-mediated cytotoxicity, the ability to grow T cells has factored heavily in the choice of cell type for most trials. The method described herein avoids the complexity and major expense related to growing T cells ex vivo or genetically modifying T cells. Multiple types of effector cells can be armed since they are readily available from peripheral blood and do not require ex vivo growth expansion.

Therefore, the anti-neoantigen antibodies described herein may be coated onto effector cells. The resulting effector cells are referred to as “armed effector cells.” Armed effector cells may comprise any number of anti-neoantigen antibodies, such as 1, 2, 3, 4, 5, 6, 7, 8, 9, 10, 11, 12, 13, 14, 15, 16, 17, 18, 19, 20, or more anti-neoantigen antibodies. In one embodiment, the effector cells comprise nine anti-neoantigen antibodies. In another embodiment, the effector cells comprise four anti-neoantigen antibodies. Examples of effector cells include, but are not limited to white blood cells (leukocytes), such as natural killer (NK) cells, neutrophils, T cells, B cells, and monocytes/macrophages. In some embodiments, a single type of effector cell is used. In other embodiments, a combination of effector cells is used. The effector cells may be coated (“armed”) with the antibodies using any method known in the art. For example, the cells may be incubated on ice with an anti-neoantigen antibody (or cocktail of anti-neoantigen antibodies).

The disclosure, in some aspects, provides a composition comprising two or more anti-neoantigen antibodies (or effector cells comprising anti-neoantigen antibodies) and a pharmaceutically acceptable carrier (excipient). In some embodiments, the composition comprises two or more anti-neoantigen antibodies, for instance, 2, 3, 4, 5, 6, 7, 8, 9, 10, 11, 12, 13, 14, 15, 16, 17, 18, 19, 20, or more anti-neoantigen antibodies. In one embodiment, the composition comprises nine anti-neoantigen antibodies. In some embodiments, the two or more anti-neoantigen antibodies are present in the composition in equal concentrations. In other embodiments, the two or more anti-neoantigen antibodies are not present in the composition in equal concentrations. In some embodiments, the at least two anti-neoantigen antibodies are all directed to the same neoantigen. In some embodiments, the at least two anti-neoantigen antibodies are directed to different epitopes of the same neoantigen. In other embodiments, the at least two anti-neoantigen antibodies are not all directed to the same neoantigen. In another embodiment, the at least two anti-neoantigen antibodies are all directed to different neoantigens.

In other aspects, the disclosure provides immune checkpoint inhibitors, for use in combination with the anti-neoantigen antibodies (or effector cells comprising anti-neoantigen antibodies) described herein.

Inhibitory checkpoint molecules include, but are not limited to: PD-1, PD-L1, PD-L2, TIM-3, VISTA, A2AR, B7-H3, B7-H4, B7-H6, BTLA, CTLA-4, IDO, KIR and LAG3. CTLA-4, PD-1, and ligands thereof are members of the CD28-B7 family of co-signaling molecules that play important roles throughout all stages of T-cell function and other cell functions. CTLA-4, Cytotoxic T-Lymphocyte-Associated protein 4 (CD152), is involved in controlling T cell proliferation.

The PD-1 receptor is expressed on the surface of activated T cells (and B cells) and, under normal circumstances, binds to its ligands (PD-L1 and PD-L2) that are expressed on the surface of antigen-presenting cells, such as dendritic cells or macrophages. This interaction sends a signal into the T cell and inhibits it. Cancer cells take advantage of this system by driving high levels of expression of PD-L1 on their surface. This allows cancer cells to gain control of the PD-1 pathway and switch off T cells expressing PD-1 that may enter the tumor microenvironment, thus suppressing the anticancer immune response. Pembrolizumab (formerly MK-3475 and lambrolizumab, trade name KEYTRUDA®) is a human antibody used in cancer immunotherapy and targets the PD-1 receptor.

The checkpoint inhibitor, in some embodiments, is a molecule such as a monoclonal antibody, a humanized antibody, a fully human antibody, a fusion protein or a combination thereof or a small molecule. For instance, the checkpoint inhibitor inhibits a checkpoint protein which may be CTLA-4, PDL1, PDL2, PD1, B7-H3, B7-H4, B7-H6, BTLA, HVEM, TIM3, GAL9, LAG3, VISTA, KIR, 2B4, CD160, CGEN-15049, CHK 1, CHK2, A2aR, B-7 family ligands or a combination thereof. Ligands of checkpoint proteins include but are not limited to CTLA-4, PDL1, PDL2, PD1, B7-H3, B7-H4, BTLA, HVEM, TIM3, GAL9, LAG3, VISTA, KIR, 2B4, CD160, CGEN-15049, CHK 1, CHK2, A2aR, and B-7 family ligands. In some embodiments the anti-PD-1 antibody is BMS-936558 (nivolumab). In other embodiments the anti-CTLA-4 antibody is ipilimumab (trade name Yervoy, formerly known as MDX-010 and MDX-101). In another embodiment, the checkpoint inhibitor is J43 (an anti-PD1 antibody), RMP1-14 (an anti-PD1 antibody), or atezolizumab (TECENTRIQ®; an anti-PDL1 antibody). In yet other embodiments, the checkpoint inhibitor is pembrolizumab.

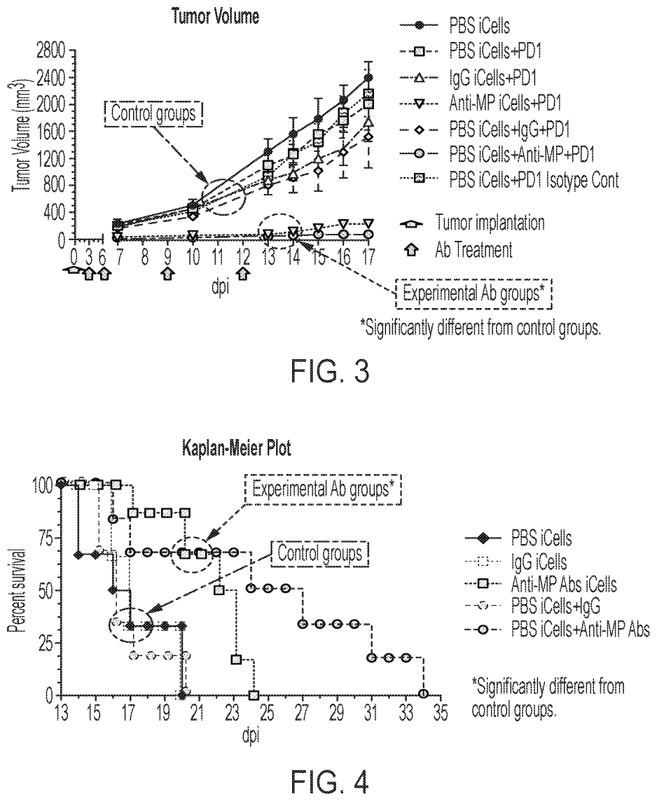

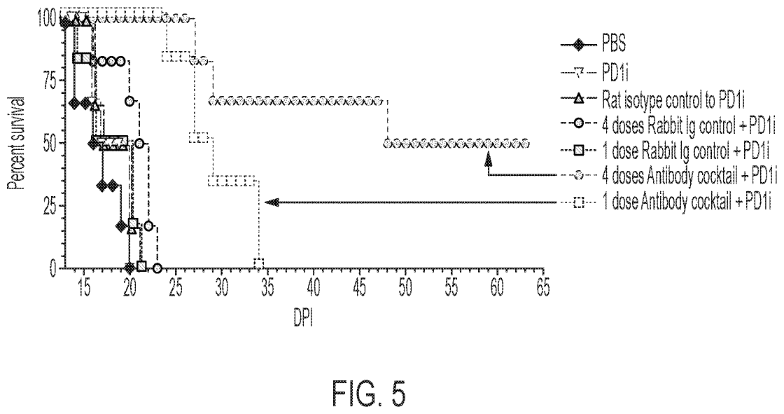

Pembrolizumab is a potent humanized immunoglobulin G4 monoclonal antibody with high specificity of binding to the PD-1 receptor, thus inhibiting its interaction with PD-L1 and programmed cell death 1 ligand 2. Based on preclinical in vitro data, pembrolizumab has high affinity and potent receptor blocking activity for PD-1. Pembrolizumab has an acceptable preclinical safety profile and is in clinical development as an IV immunotherapy for advanced malignancies. KEYTRUDA® (pembrolizumab) is approved for the treatment of patients across a number of indications. Pembrolizumab is approved for use in several cancer types, and is under investigation in several phases of clinical development for many more. Despite much progress in the field of immune-oncology therapeutics, not all subjects respond to pembrolizumab therapy, most responses are not complete, and it is only approved for use in limited tumor types. Combining pembrolizumab with the anti-neoantigen antibodies disclosed herein may allow more subjects to derive greater clinical benefit than with pembrolizumab monotherapy (FIGS. 3 and 5).

In some embodiments, the compositions and methods further comprise administering at least one immune checkpoint inhibitor, as described herein. In some embodiments, combinations of immune checkpoint inhibitors are administered.

Provided herein, in some aspects, is a method of treating a cancer, the method comprising administering to a subject having cancer, two or more anti-neoantigen antibodies and an immune checkpoint inhibitor, in an effective amount to treat the cancer. In some embodiments, the two or more anti-neoantigen antibodies and the immune checkpoint inhibitor are administered simultaneously. In other embodiments, the two or more anti-neoantigen antibodies are administered to the subject prior to administration of the immune checkpoint inhibitor. In some embodiments, the two or more anti-neoantigen antibodies are administered in separate formulations to the subject. In other embodiments, the two or more anti-neoantigen antibodies are administered in the same formulation to the subject.

In an additional aspect, the disclosure provides a method of treating a cancer, the method comprising administering to a subject having cancer, effector cells comprising anti-neoantigen antibodies and an immune checkpoint inhibitor, in an effective amount to treat the cancer. In some embodiments, the effector cells comprising anti-neoantigen antibodies and the immune checkpoint inhibitor are administered simultaneously. In other embodiments, the effector cells comprising anti-neoantigen antibodies are administered to the subject prior to administration of the immune checkpoint inhibitor.

As used herein, the term “treating” or “treatment” refers to the application or administration of a composition including one or more active agents to a subject, who has a target disease or disorder (e.g., cancer) or a symptom of the disease/disorder with the purpose to prevent, cure, heal, alleviate, relieve, alter, remedy, ameliorate, improve, or affect the disorder or the symptom of the disease.

Alleviating a target disease/disorder (e.g., cancer) includes delaying the development or progression of the disease, or reducing disease severity or prolonging survival. Alleviating the disease or prolonging survival does not necessarily require curative results. As used therein, “delaying” the development of a target disease or disorder means to defer, hinder, slow, retard, stabilize, and/or postpone progression of the disease. This delay can be of varying lengths of time, depending on the history of the disease and/or individuals being treated. A method that “delays” or alleviates the development of a disease, or delays the onset of the disease, is a method that reduces probability of developing one or more symptoms of the disease in a given time frame and/or reduces extent of the symptoms in a given time frame, when compared to not using the method. Such comparisons are typically based on clinical studies, using a number of subjects sufficient to give a statistically significant result.

“Development” or “progression” of a disease means initial manifestations and/or ensuing progression of the disease. Development of the disease can be detectable and assessed using standard clinical techniques as well known in the art. However, development also refers to progression that may be undetectable. For purpose of this disclosure, development or progression refers to the biological course of the symptoms. “Development” includes occurrence, recurrence, and onset. As used herein “onset” or “occurrence” of a target disease or disorder includes initial onset and/or recurrence.

Cancers or tumors include but are not limited to neoplasms, malignant tumors, metastases, or any disease or disorder characterized by uncontrolled cell growth such that it would be considered cancerous. The cancer may be a primary or metastatic cancer. Specific cancers that can be treated according to the present disclosure include, but are not limited to, those listed below (for a review of such disorders, see Fishman et al., 1985, Medicine, 2d Ed., J.B. Lippincott Co., Philadelphia). Cancers for use with the instantly described methods and compositions may include, but are not limited to, basal cell carcinoma, bladder cancer, bone cancer, bowel carcinoma, breast cancer, carcinoid, anal squamous cell carcinoma, castration-resistant prostate cancer (CRPC), cervical carcinoma, colorectal cancer (CRC), colon cancer cutaneous squamous cell carcinoma, endometrial cancer, esophageal cancer, gastric carcinoma, gastroesophageal junction cancer, glioblastoma/mixed glioma, glioma, head and neck cancer, hepatocellular carcinoma, hematologic malignancy. liver cancer, lung cancer, melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer, rectal carcinoma, renal cancer, sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, and uterine sarcoma.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. In one embodiment, the composition is administered via intratumoral injection. In a specific embodiment, the composition is administered via intraperitoneal injection. In addition, it can be administered to the subject via injectable depot routes of administration such as using 1-, 3-, or 6-month depot injectable or biodegradable materials and methods. In some examples, the pharmaceutical composition is administered intraocularly or intravitreally.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the anti-neoantigen cocktail or effector cells comprising anti-neoantigen antibodies and at least one immune checkpoint inhibitor and a physiologically acceptable excipient is infused. Physiologically acceptable excipients may include, for example, 5% dextrose, 0.9% saline, Ringer's solution or other suitable excipients. Intramuscular preparations, e.g., a sterile formulation of a suitable soluble salt form of the anti-neoantigen antibody, anti-neoantigen antibody cocktail, or effector cells comprising anti-neoantigen antibodies, can be dissolved and administered in a pharmaceutical excipient such as Water-for-Injection, 0.9% saline, or 5% glucose solution.

In one embodiment, a composition is administered via site-specific or targeted local delivery techniques. Examples of site-specific or targeted local delivery techniques include various implantable depot sources of the composition or local delivery catheters, such as infusion catheters, an indwelling catheter, or a needle catheter, synthetic grafts, adventitial wraps, shunts and stents or other implantable devices, site specific carriers, direct injection, or direct application. See, e.g., PCT Publication No. WO 00/53211 and U.S. Pat. No. 5,981,568.

The particular dosage regimen, i.e., dose, timing and repetition, used in the method described herein will depend on the particular subject and that subject's medical history. In some embodiments, the two or more anti-neoantigen antibodies are administered to the subject at least 1, 2, 3, 4, 5, 6, 7, 8, 9, 10, 11, 12, 13, 14, 15, 16, 17, 18, 19, 20, or more times. In one embodiment, the two or more anti-neoantigen antibodies are administered to the subject at least twice. In another embodiment, the two or more anti-neoantigen antibodies are administered to the subject at least four times.

In some embodiments, the effector cells comprising anti-neoantigen antibodies are administered to the subject at least 1, 2, 3, 4, 5, 6, 7, 8, 9, 10, 11, 12, 13, 14, 15, 16, 17, 18, 19, 20, or more times. In one embodiment, the effector cells comprising anti-neoantigen antibodies are administered to the subject at least twice. In another embodiment, the effector cells comprising anti-neoantigen antibodies are administered to the subject at least five times.

In some embodiments, the immune checkpoint inhibitor is an anti-CTLA-4 antibody. In one embodiment, the immune checkpoint inhibitor is the anti-CTLA-4 antibody ipilimumab. Optionally, the ipilimumab is administered to the subject at a dose of about 3 mg/kg to 10 mg/kg or a fixed dose of about 240 mg to 800 mg. Other dosages are also possible. For example, the dose may be 1, 2, 3, 4, 5, 6, 7, 8, 9, 10, 11, 12, 13, 14, 15, 16, 17, 18, 19, 20 mg/kg or more. The fixed dose may be 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000 mg or more.

Likewise, in some embodiments, the immune checkpoint inhibitor is an anti-PD1 antibody. In one embodiment, the anti-PD1 antibody is pembrolizumab. Optionally, the pembrolizumab is administered to the subject at a dose of about 3 mg/kg to 10 mg/kg or a fixed dose of about 240 mg to 800 mg. Other dosages are also possible. For example, the dose may be 1, 2, 3, 4, 5, 6, 7, 8, 9, 10, 11, 12, 13, 14, 15, 16, 17, 18, 19, 20 mg/kg or more. The fixed dose may be 100, 120, 140, 160, 180, 200, 220, 240, 260, 280, 300, 320, 340, 360, 380, 400, 420, 440, 460, 480, 500, 520, 540, 560, 580, 600, 620, 640, 660, 680, 700, 720, 740, 760, 780, 800, 820, 840, 860, 880, 900, 920, 940, 960, 980, 1000 mg or more.

In some embodiments, the immune checkpoint inhibitor is administered on a schedule of one dose aver 7-30 days, for example, one dose every 7, 8, 9, 10, 11, 12, 13, 14, 15, 16, 17, 18, 19, 20, 21, 22, 23, 24, 25, 26, 27, 28, 29, or 30 days. In one particular embodiment, the immune checkpoint inhibitor is administered as one dose every 14 days. In another specific embodiment, the immune checkpoint inhibitor is administered as one dose every 21 days.

As shown in FIG. 2, in some embodiments, the two or more anti-neoantigen antibodies produce a significant reduction in tumor volume, for example, relative to controls treated with PBS or an isotype antibody. In some embodiments, the two or more anti-neoantigen antibodies and checkpoint inhibitor produce a significant reduction in tumor volume, for example, relative to administration of the antibodies or the immune checkpoint inhibitor alone (FIG. 3). As shown in FIG. 4, in some embodiments, the two or more anti-neoantigen antibodies produce a significant increase in survival rate, for example, relative to controls treated with PBS or an isotype antibody. In some embodiments, the two or more anti-neoantigen antibodies and checkpoint inhibitor produce a significant increase in survival rate, for example, relative to administration of the antibodies or immune checkpoint alone (FIG. 5).

In some embodiments, the two or more anti-neoantigen antibodies produce a durable immune response; that is, when the subject is exposed to the antigen (e.g., cancer) one or more subsequent times, the subject mounts an effective anti-cancer immune response. In some embodiments, the durable immune response persists for 1 day, 2 days, 3 days, 4 days, 5 days, 6 days, 1 week, 1.5 weeks, 2 weeks, 2.5 weeks, 3 weeks, 3.5 weeks, 1 month, 2 months, 3 months, 4 months, 5 months, 6 months, 7 months, 8 months, 9 months, 10 months, 11 months, 1 year, 1.5 years, 2 years, or longer, following an initial exposure to the antigen (e.g., cancer) and treatment with any of the compositions described herein (i.e., Example 1, Experiment 4). In some embodiments, the durable immune response persists for at least 1 day, at least 2 days, at least 3 days, at least 4 days, at least 5 days, at least 6 days, at least 1 week, at least 1.5 weeks, at least 2 weeks, at least 2.5 weeks, at least 3 weeks, at least 3.5 weeks, at least 1 month, at least 2 months, at least 3 months, at least 4 months, at least 5 months, at least 6 months, at least 7 months, at least 8 months, at least 9 months, at least 10 months, at least 11 months, at least 1 year, at least 1.5 years, at least 2 years, or longer, following an initial exposure to the antigen (e.g., cancer) and treatment with any of the compositions described herein. In one embodiment, the durable immune response persists for at least six months.

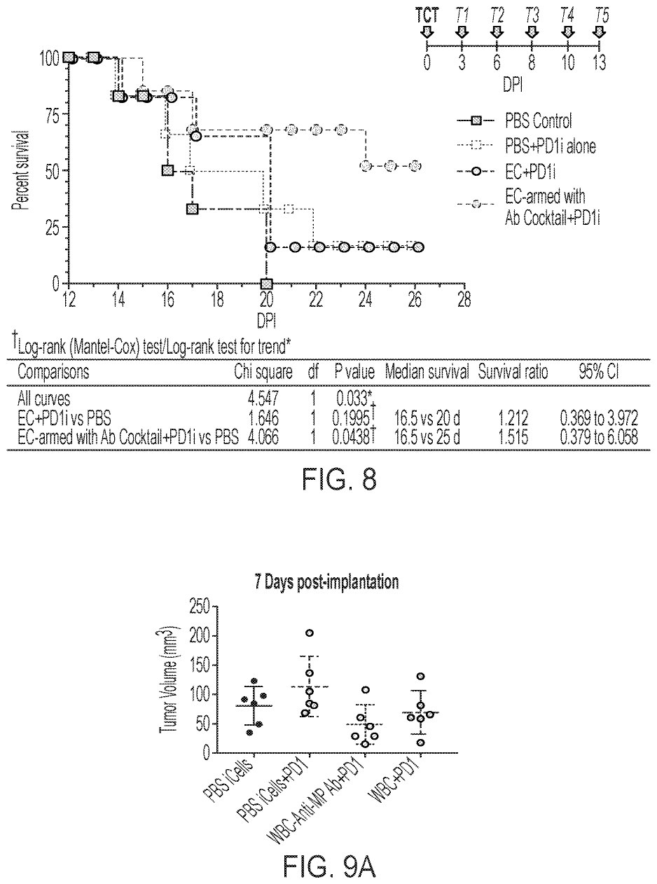

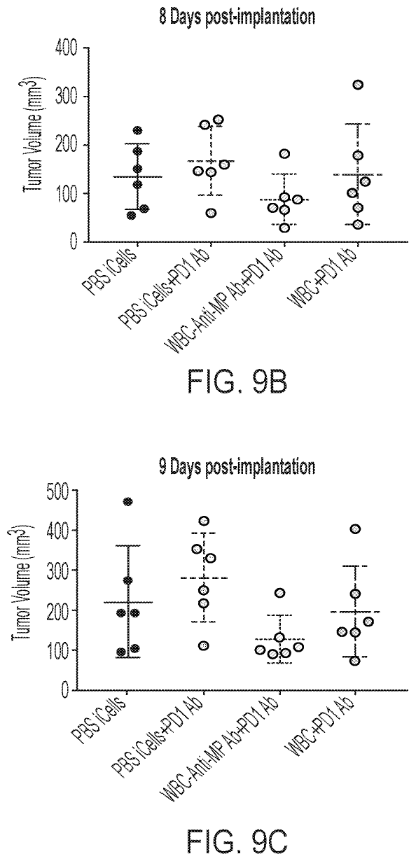

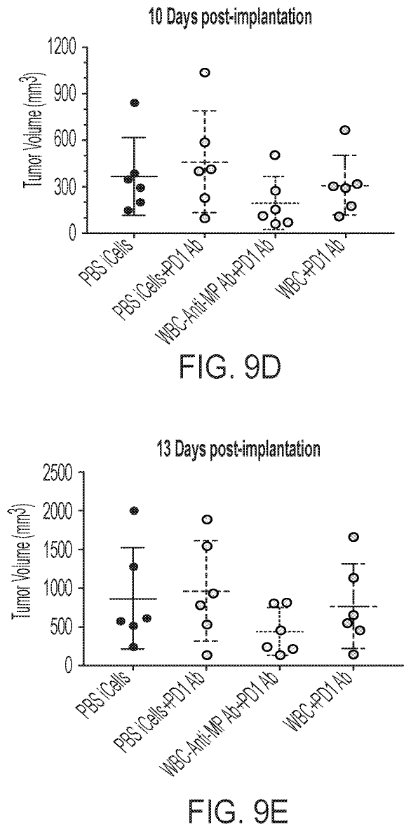

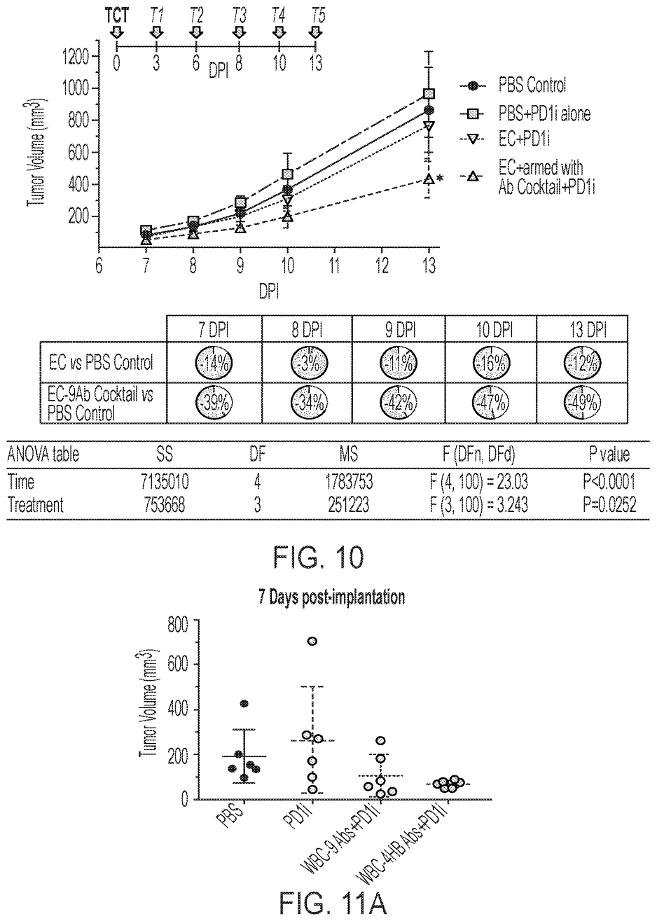

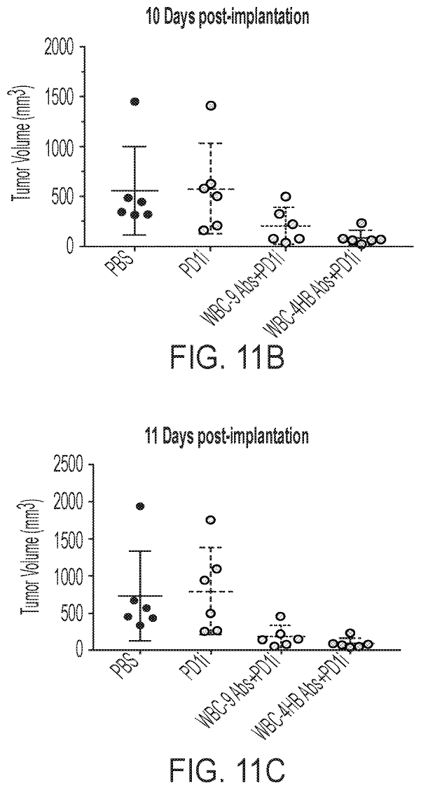

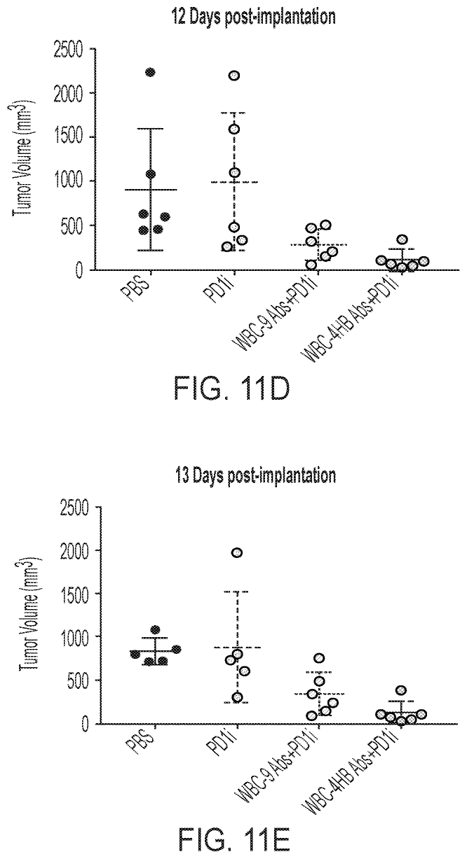

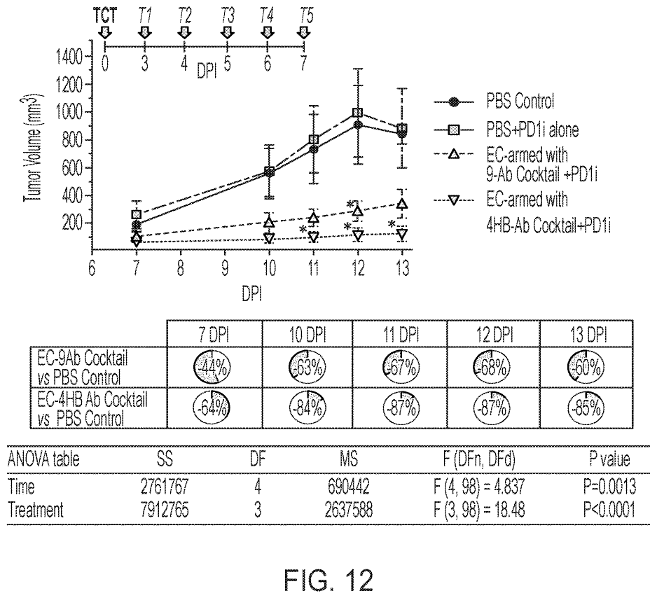

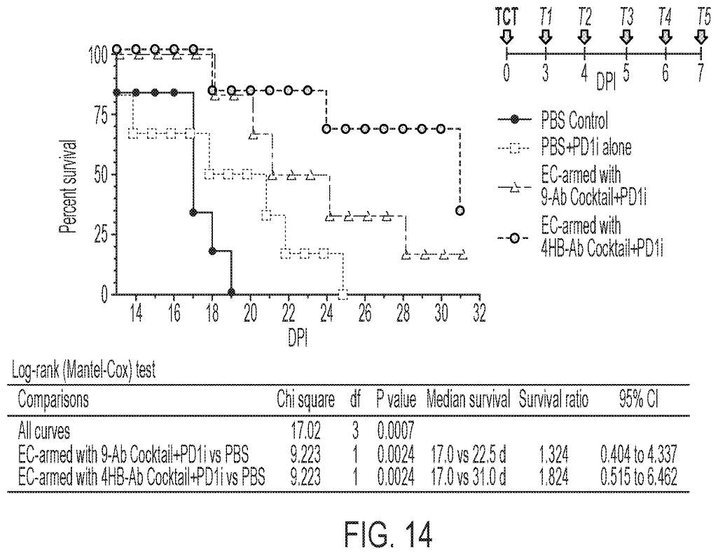

FIG. 10 demonstrates that, in some embodiments, the effector cells comprising anti-neoantigen antibodies and checkpoint inhibitor, produce a significant reduction in tumor volume, for example, relative to controls treated with PBS, treated with PBS and a PD-1 inhibitor, or treated with effector cells (without anti-neoantigen antibodies) and a PD-1 inhibitor. The effect was even more drastic when the effector cells only comprised the four high binding anti-neoantigen antibodies (FIG. 12). In some embodiments, the effector cells comprising anti-neoantigen antibodies and checkpoint inhibitor produce a significant increase in survival rate, for example, relative to controls treated with PBS, treated with PBS and a PD-1 inhibitor, or treated with effector cells (without anti-neoantigen antibodies) and a PD-1 inhibitor (FIG. 8). The effect was also more drastic when the effector cells only comprised the four high binding anti-neoantigen antibodies (FIG. 14).

In other embodiments, the subject is non-responsive to immune checkpoint inhibitor therapy. In some embodiments, tumor mutation burden (TMB), the total number of non-synonymous somatic mutations of the genomic coding area, which may be determined using whole exome sequencing, next-generation sequencing, or other methods known in the art, may be predictive of a subject's responsiveness to immune checkpoint inhibitor therapy. In addition or alternatively, the level of one or more biomarkers, such as PD-L1, may be predictive of the subject's responsiveness of immune checkpoint inhibitor therapy. High mutation loads and certain somatic neoepitopes (e.g., those resulting from tumor mutations) may also correlate with responsiveness. Further, elevated histone H3 lysine (27) trimethylation (H3K27me3 and decreased E-cadherin may be indicative of resistance to immune checkpoint inhibitor therapy (Shields et al., Scientific Reports, 2017, 7: 807). In one embodiment, the subject does not have a favorable response to the immune checkpoint therapy based on RECIST (Response Evaluation Criteria In Solid Tumors), irRECIST (Immune-related Response Evaluation Criteria In Solid Tumors), or iRECIST. iRECIST and irRECIST, which are adapted from RECIST, account for the unique tumor response seen with immunotherapeutic drugs and is therefore used to assess tumor response and progression, and make treatment decisions. In another embodiment, if the subject was previously administered one or more immune checkpoint inhibitors, but the subject's symptoms or disease did not improve and/or progressed still further, the subject may be non-responsive to immune checkpoint inhibitor therapy.

In some embodiments, more than one composition, or a combination of a composition described herein and another suitable therapeutic agent, may be administered to a subject in need of the treatment. The composition described herein can also be used in conjunction with other agents that serve to enhance and/or complement the effectiveness of the agents.

In still further embodiments, the method may further comprise administering an anti-cancer agent. Examples of anti-cancer agents include, but are not limited to, cancer vaccines, chemotherapy, radiation, and immunotherapeutics (e.g., modified T cell therapy). In one embodiment, the anti-neoantigen antibodies can be incorporated into chimeric antigen receptors (CARs) and then expressed on the surface of immune effector cells (e.g., T cells). CARs, generally, are artificial immune cell receptors engineered to recognize and bind to an antigen expressed by tumor cells. CARs typically include an antibody fragment as an antigen-binding domain (e.g., an anti-neoantigen antibody), a hydrophobic alpha helix transmembrane domain, and one or more intracellular signaling/co-signaling domains, such as (but not limited to) CD3-zeta, CD28, 4-1BB and/or OX40. A CAR may include a signaling domain or at least two co-signaling domains. In some embodiments, a CAR includes three or four co-signaling domains. Generally, a CAR is designed for a T cell and is a chimera of a signaling domain of the T-cell receptor (TcR) complex and an antigen-recognizing domain (e.g., a single chain fragment (scFv) of an antibody, e.g., an anti-ncoantigen antibody of the instant disclosure) (Enblad et al., Human Gene Therapy. 2015; 26(8):498-505). A T cell that expresses a CAR is known in the art as a CAR T cell. As an example, a CAR construct having an extracellular domain comprising one or more of the anti-neoantigen antibodies of the present disclosure (e.g., an anti-neoantigen antibody in CAR format), a transmembrane domain, and an intracellular signaling domain may be generated. The CAR construct can then be used to transfect T cells removed from the blood of a subject, thereby producing a functional CAR in the T cells. The resulting CAR T cells may then be administered to the subject, where they target tumor cells. In some embodiments, an anti-neoantigen antibody in CAR format is administered with effector cells comprising anti-neoantigen antibodies. In some embodiments, the CAR T cell comprises the same anti-neoantigen antibodies as the effector cells comprising anti-ncoantigen antibodies. In other embodiments, the CAR T cell comprises different anti-neoantigen antibodies from the anti-neoantigen antibodies arming the effector cells.

Thus, in one embodiment, the methods of the disclosure can be used in conjunction with one or more cancer therapeutics, for example, in conjunction with an anti-cancer agent, a traditional cancer vaccine, chemotherapy, radiotherapy, etc. (e.g., simultaneously, or as part of an overall treatment procedure). Parameters of cancer treatment that may vary include, but are not limited to, dosages, timing of administration or duration or therapy; and the cancer treatment can vary in dosage, timing, or duration. Another treatment for cancer is surgery, which can be utilized either alone or in combination with any of the previous treatment methods. Any agent or therapy (e.g., traditional cancer vaccines, chemotherapies, radiation therapies, surgery, hormonal therapies, and/or biological therapies/immunotherapies) which is known to be useful, or which has been used or is currently being used for the prevention or treatment of cancer can be used in combination with a composition of the disclosure in accordance with the disclosure described herein. One of ordinary skill in the medical arts can determine an appropriate treatment for a subject.

Examples of such agents (i.e., anti-cancer agents) include, but are not limited to, DNA-interactive agents including, but not limited to, the alkylating agents (e.g., nitrogen mustards, e.g., Chlorambucil, Cyclophosphamide, Isofamide, Mechlorethamine, Melphalan, Uracil mustard; Aziridine such as Thiotepa; methanesulphonate esters such as Busulfan; nitroso ureas, such as Carmustine, Lomustine, Streptozocin; platinum complexes, such as Cisplatin, Carboplatin; bioreductive alkylator, such as Mitomycin, and Procarbazine, Dacarbazine and Altretamine); the DNA strand-breakage agents, e.g., Bleomycin; the intercalating topoisomerase TT inhibitors, e.g., Intercalators, such as Amsacrine, Dactinomycin, Daunorubicin, Doxorubicin, Idarubicin, Mitoxantrone, and nonintercalators, such as Etoposide and Teniposide; the nonintercalating topoisomerase II inhibitors, e.g., Etoposide and Teniposde; and the DNA minor groove binder, e.g., Plicamydin; the antimetabolites including, but not limited to, folate antagonists such as Methotrexate and trimetrexate; pyrimidine antagonists, such as Fluorouracil, Fluorodeoxyuridine, CB3717, Azacitidine and Floxuridine; purine antagonists such as Mercaptopurine, 6-Thioguanine, Pentostatin; sugar modified analogs such as Cytarabine and Fludarabine; and ribonucleotide reductase inhibitors such as hydroxyurea; tubulin interactive agents including, but not limited to, colcbicine, Vincristine and Vinblastine, both alkaloids and Paclitaxel and cytoxan; hormonal agents including, but not limited to, estrogens, conjugated estrogens and Ethinyl Estradiol and Diethylstilbesterol, Chlortrianisen and Idenestrol; progestins such as Hydroxyprogesterone caproate, Medroxyprogesterone, and Megestrol; and androgens such as testosterone, testosterone propionate; fluoxymesterone, methyltestosterone; adrenal corticosteroid, e.g., Prednisone, Dexamethasone, Methylprednisolone, and Prednisolone; leutinizing hormone releasing hormone agents or gonadotropin-releasing hormone antagonists, e.g., leuprolide acetate and goserelin acetate; antihormonal antigens including, but not limited to, antiestrogenic agents such as Tamoxifen, antiandrogen agents such as Flutamide; and antiadrenal agents such as Mitotane and Aminoglutethimide; cytokines including, but not limited to, IL-1.alpha., IL-1β, IL-2, IL-3, IL-4, IL-5, IL-6, IL-7, IL-8, IL-9, IL-10, IL-11, IL-12, IL-13, IL-18, TGF-β, GM-CSF, M-CSF, G-CSF, TNF-α, TNF-β, LAF, TCGF, BCGF, TRF, BAF, BDG, MP, LIF, OSM, TMF, PDGF, IFN-α, TFN-β, IFN-γ, and Uteroglobins (U.S. Pat. No. 5,696,092); anti-angiogenics including, but not limited to, agents that inhibit VEGF (e.g., other neutralizing antibodies), soluble receptor constructs, tyrosine kinase inhibitors, antisense strategies, RNA aptamers and ribozymes against VEGF or VEGF receptors, immunotoxins and coaguligands, tumor vaccines, and antibodies.

Specific examples of anti-cancer agents which can be used in accordance with the methods of the disclosure include, but not limited to: acivicin; aclarubicin; acodazole hydrochloride; acronine; adozelesin; aldesleukin; altretamine; ambomycin; ametantrone acetate; aminoglutethimide; amsacrine; anastrozole; anthramycin; asparaginase; asperlin; azacitidine; azetepa; azotomycin; batimastat; benzodepa; bicalutamide; bisantrene hydrochloride; hisnafide dimesylate; bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine; dacarbazine; dactinomycin; daunorubicin hydrochloride; decitabine; dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; flurocitabine; fosquidone; fostriecin sodium; gemcitabine; gemcitabine hydrochloride; hydroxyurea; idarubicin hydrochloride; ifosfamide; ilmofosine; interleukin II (including recombinant interieukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; letrozole; leuprolide acetate; liarozole hydrochloride; lometrexol sodium; lomustine; losoxantrone hydrochloride; masoprocol; maytansine; mechlorethamine hydrochloride; megestrol acetate; melengestrol acetate; melphalan; menogaril; mercaptopurine; methotrexate; methotrexate sodium; metoprine; meturedepa; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; and zorubicin hydrochloride.

Other anti-cancer drugs which may be used with the instant compositions and methods include, but are not limited to: 20-epi-1,25 dihydroxyvitamin D3; 5-ethynyluracil; angiogenesis inhibitors; anti-dorsalizing morphogenetic protein-1; ara-CDP-DL-PTBA; BCR/ABL antagonists; CaRest M3; CARN 700; casein kinase inhibitors (ICOS); clotrimazole; collismycin A; collismycin B; combretastatin A4; crambescidin 816; cryptophycin 8; curacin A; dehydrodidemnin B; didemnin B; dihydro-5-azacytidine; dihydrotaxol, duocarmycin SA; kahalalide F; lamellarin-N triacetate; leuprolide+estrogen+progesterone; lissoclinamide 7; monophosphoryl lipid A+myobacterium cell wall sk; N-acetyldinaline; N-substituted benzamides; 06-benzylguanine; placetin A; placetin B; platinum complex; platinum compounds; platinum-triamine complex; rhenium Re 186 etidronate; RII retinamide; rubiginone B 1; SarCNU; sarcophytol A; sargramostim; senescence derived inhibitor 1; spicamycin D; tallimustine; 5-fluorouracil; thrombopoietin; thymotrinan; thyroid stimulating hormone; variolin B; thalidomide; velaresol; veramine; verdins; verteporfin; vinorelbine; vinxaltine; vitaxin; zanoterone; zeniplatin; and zilascorb.

The disclosure also encompasses administration of a composition comprising two or more anti-neoantigen antibodies (or effector cells comprising anti-neoantigen antibodies) and at least one immune checkpoint inhibitor in combination with radiation therapy comprising the use of x-rays, gamma rays and other sources of radiation to destroy the cancer cells. In certain embodiments, the radiation treatment is administered as external beam radiation or teletherapy wherein the radiation is directed from a remote source. In other embodiments, the radiation treatment is administered as internal therapy or brachytherapy wherein a radioactive source is placed inside the body close to cancer cells or a tumor mass.

In specific embodiments, an appropriate anti-cancer regimen is selected depending on the type of cancer (e.g., by a physician). For instance, a patient with ovarian cancer may be administered a prophylactically or therapeutically effective amount of a composition comprising anti-neoantigen antibodies (or effector cells comprising anti-neoantigen antibodies) and, optionally, at least one immune checkpoint inhibitor, in combination with a prophylactically or therapeutically effective amount of one or more other agents useful for ovarian cancer therapy, including but not limited to, intraperitoneal radiation therapy, such as P32 therapy, total abdominal and pelvic radiation therapy, cisplatin, the combination of paclitaxel (Taxol) or docetaxel (Taxotere) and cisplatin or carboplatin, the combination of cyclophosphamide and cisplatin, the combination of cyclophosphamide and carboplatin, the combination of 5-FU and leucovorin, etoposide, liposomal doxorubicin, gemcitabine or topotecan. Cancer therapies and their dosages, routes of administration and recommended usage are known in the art and have been described in such literature as the Physician's Desk Reference (56th ed., 2002).

In some embodiments the cancer therapeutic agent is a targeted therapy. The targeted therapy may be a BRAF inhibitor such as vemurafenib (PLX4032) or dabrafenib. The BRAF inhibitor may be PLX 4032, PLX 4720, PLX 4734, GDC-0879, PLX 4032, PLX-4720, PLX 4734 and Sorafenib Tosylate. BRAF is a human gene that makes a protein called B-Raf, also referred to as proto-oncogene B-Raf and v-Raf murine sarcoma viral oncogene homolog B1. The B-Raf protein is involved in sending signals inside cells, which are involved in directing cell growth. Vemurafenib, a BRAF inhibitor, was approved by FDA for treatment of late-stage melanoma.

In other embodiments the cancer therapeutic agent is a cytokine. In yet other embodiments the cancer therapeutic agent is a vaccine comprising a population-based tumor specific antigen. In yet other embodiments, the cancer therapeutic agent is vaccine containing one or more traditional antigens expressed by cancer-germline genes (antigens common to tumors found in multiple patients, also referred to as “shared cancer antigens”). In some embodiments, a traditional antigen is one that is known to be found in cancers or tumors generally or in a specific type of cancer or tumor. In some embodiments, a traditional cancer antigen is a non-mutated tumor antigen. In some embodiments, a traditional cancer antigen is a mutated tumor antigen.

Provided herein, in some aspects is a composition comprising two or more anti-neoantigen antibodies (or effector cells comprising anti-neoantigen antibodies) and a pharmaceutically acceptable carrier (excipient). Optionally, the composition may further comprise one or more immune checkpoint inhibitors. “Acceptable” means that the carrier must be compatible with the active ingredient of the composition (and preferably, capable of stabilizing the active ingredient) and not deleterious to the subject to be treated. Pharmaceutically acceptable excipients (carriers) including buffers, which are well known in the art. See, e.g., Remington: The Science and Practice of Pharmacy 20th Ed. (2000) Lippincott Williams and Wilkins, Ed. K. E. Hoover.

The pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover). Acceptable carriers, excipients, or stabilizers are nontoxic to recipients at the dosages and concentrations used, and may comprise buffers such as phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrans; chelating agents such as EDTA; sugars such as sucrose, mannitol, trehalose or sorbitol; salt-forming counter-ions such as sodium; metal complexes (e.g. Zn-protein complexes); and/or non-ionic surfactants such as TWEEN™, PLURONICS™ or polyethylene glycol (PEG).

In some examples, the pharmaceutical composition described herein comprises liposomes containing the antibodies which can be prepared by methods known in the art, such as described in Epstein, et al., Proc. Natl. Acad. Sci. USA 82:3688 (1985); Hwang, et al., Proc. Natl. Acad. Sci. USA 77:4030 (1980); and U.S. Pat. Nos. 4,485,045 and 4,544,545. Liposomes with enhanced circulation time are disclosed in U.S. Pat. No. 5,013,556. Particularly useful liposomes can be generated by the reverse phase evaporation method with a lipid composition comprising phosphatidylcholine, cholesterol and PEG-derivatized phosphatidylethanolamine (PEG-PE). Liposomes are extruded through filters of defined pore size to yield liposomes with the desired diameter.

The antibodies may also be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are known in the art, see, e.g., Remington, The Science and Practice of Pharmacy 20th Ed. Mack Publishing (2000).

In other examples, the pharmaceutical composition described herein can be formulated in sustained-release format. Suitable examples of sustained-release preparations include semipermeable matrices of solid hydrophobic polymers containing the antibody, which matrices are in the form of shaped articles, e.g. films, or microcapsules. Examples of sustained-release matrices include polyesters, hydrogels (for example, poly(2-hydroxyethyl-methacrylate), or poly(vinyl alcohol)), polylactides (U.S. Pat. No. 3,773,919), copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

The pharmaceutical compositions to be used for in vivo administration must be sterile. This is readily accomplished by, for example, filtration through sterile filtration membranes. Therapeutic antibody compositions are generally placed into a container having a sterile access port, for example, an intravenous solution bag or vial having a stopper pierceable by a hypodermic injection needle.

The pharmaceutical compositions described herein can be in unit dosage forms such as tablets, pills, capsules, powders, granules, solutions or suspensions, or suppositories, for oral, parenteral or rectal administration, or administration by inhalation or insufflation.

For preparing solid compositions such as tablets, the principal active ingredient can be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the present invention, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules. This solid preformulation composition is then subdivided into unit dosage forms of the type described above containing from 0.1 to about 500 mg of the active ingredient of the present invention. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

Suitable surface-active agents include, in particular, non-ionic agents, such as polyoxyethylenesorbitans (e.g., Tween™ 20, 40, 60, 80 or 85) and other sorbitans (e.g., Span™ 20, 40, 60, 80 or 85). Compositions with a surface-active agent will conveniently comprise between 0.05 and 5% surface-active agent, and can be between 0.1 and 2.5%. It will be appreciated that other ingredients may be added, for example mannitol or other pharmaceutically acceptable vehicles, if necessary.

Suitable emulsions may be prepared using commercially available fat emulsions, such as Intralipid™, Liposyn™, Infonutrol™, Lipofundin™ and Lipiphysan™. The active ingredient may be either dissolved in a pre-mixed emulsion composition or alternatively it may be dissolved in an oil (e.g., soybean oil, safflower oil, cottonseed oil, sesame oil, corn oil or almond oil) and an emulsion formed upon mixing with a phospholipid (e.g. egg phospholipids, soybean phospholipids or soybean lecithin) and water. It will be appreciated that other ingredients may be added, for example glycerol or glucose, to adjust the tonicity of the emulsion. Suitable emulsions will typically contain up to 20% oil, for example, between 5 and 20%. The fat emulsion can comprise fat droplets between 0.1 and 1.0 .im, particularly 0.1 and 0.5 .im, and have a pH in the range of 5.5 to 8.0.

The emulsion compositions can be those prepared by mixing an antibody with Intralipid™ or the components thereof (soybean oil, egg phospholipids, glycerol and water).

Pharmaceutical compositions for inhalation or insufflation include solutions and suspensions in pharmaceutically acceptable, aqueous or organic solvents, or mixtures thereof, and powders. The liquid or solid compositions may contain suitable pharmaceutically acceptable excipients as set out above. In some embodiments, the compositions are administered by the oral or nasal respiratory route for local or systemic effect.

Compositions in preferably sterile pharmaceutically acceptable solvents may be nebulized by use of gases. Nebulized solutions may be breathed directly from the nebulizing device or the nebulizing device may be attached to a face mask, tent or intermittent positive pressure breathing machine. Solution, suspension or powder compositions may be administered, preferably orally or nasally, from devices which deliver the formulation in an appropriate manner.

To practice the method disclosed herein, an effective amount of the pharmaceutical composition described herein can be administered to a subject (e.g., a human) in need of the treatment via a suitable route, such as intravenous administration, e.g., as a bolus or by continuous infusion over a period of time, by intramuscular, intraperitoneal, intracerebrospinal, subcutaneous, intra-articular, intrasynovial, intrathecal, oral, inhalation or topical routes. Commercially available nebulizers for liquid formulations, including jet nebulizers and ultrasonic nebulizers are useful for administration. Liquid formulations can be directly nebulized and lyophilized powder can be nebulized after reconstitution. Alternatively, the antibodies as described herein can be aerosolized using a fluorocarbon formulation and a metered dose inhaler, or inhaled as a lyophilized and milled powder.

The subject to be treated by the methods described herein can be a mammal, more preferably a human. Mammals include, but are not limited to, farm animals, sport animals, pets, primates, horses, dogs, cats, mice and rats. A human subject who needs the treatment may be a human patient having, at risk for, or suspected of having a target disease/disorder, such as a cancer.

A subject suspected of having any of such target disease/disorder (e.g., cancer) might show one or more symptoms of the disease/disorder. A subject at risk for the disease/disorder can be a subject having one or more of the risk factors for that disease/disorder.

As used herein, “an effective amount” refers to the amount of each active agent required to confer therapeutic effect on the subject, either alone or in combination with one or more other active agents. In some embodiments, the therapeutic effect is reduced tumor bioactivity. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In one example, dosages for an antibody as described herein may be determined empirically in individuals who have been given one or more administration(s) of the antibody. Individuals are given incremental dosages of the antagonist. To assess efficacy of the antagonist, an indicator of the disease/disorder can be followed.

Generally, for administration of any of the antibodies described herein, an initial candidate dosage can be about 2 mg/kg. For the purpose of the present disclosure, a typical daily dosage might range from about any of 0.1 μg/kg to 3 μg/kg to 30 μg/kg to 300 μg/kg to 3 mg/kg, to 30 mg/kg to 100 mg/kg or more, depending on the factors mentioned above. For repeated administrations over several days or longer, depending on the condition, the treatment is sustained until a desired suppression of symptoms occurs or until sufficient therapeutic levels are achieved to alleviate a target disease or disorder, or a symptom thereof. An exemplary dosing regimen comprises administering an initial dose of about 2 mg/kg, followed by a weekly maintenance dose of about 1 mg/kg of the antibody, or followed by a maintenance dose of about 1 mg/kg every other week. However, other dosage regimens may be useful, depending on the pattern of pharmacokinetic decay that the practitioner wishes to achieve. For example, dosing from one-four times a week is contemplated. In some embodiments, dosing ranging from about 3 μg/mg to about 2 mg/kg (such as about 3 μg/mg, about 10 μg/mg, about 30 μg/mg, about 100 μg/mg, about 300 μg/mg, about 1 mg/kg, and about 2 mg/kg) may be used. In some embodiments, dosing frequency is once every week, every 2 weeks, every 4 weeks, every 5 weeks, every 6 weeks, every 7 weeks, every 8 weeks, every 9 weeks, or every 10 weeks; or once every month, every 2 months, or every 3 months, or longer. The progress of this therapy is easily monitored by conventional techniques and assays. The dosing regimen (including the antibody used) can vary over time.

In some embodiments, for an adult patient of normal weight, doses ranging from about 0.3 to 5.00 mg/kg may be administered. In some examples, the dosage of the anti-neoantigen antibody cocktail (or effector cells comprising anti-neoantigen antibodies) described herein can be 10 mg/kg. The particular dosage regimen, i.e., dose, timing and repetition, will depend on the particular individual and that individual's medical history, as well as the properties of the individual agents (such as the half-life of the agent, and other considerations well known in the art).

For the purpose of the present disclosure, the appropriate dosage of an antibody as described herein will depend on the specific antibody, antibodies, and/or non-antibody peptide (or compositions thereof) employed, the type and severity of the disease/disorder, whether the antibody is administered for preventive or therapeutic purposes, previous therapy, the patient's clinical history and response to the antagonist, and the discretion of the attending physician. Typically the clinician will administer an antibody, until a dosage is reached that achieves the desired result. In some embodiments, the desired result is an increase in anti-tumor immune response in the tumor microenvironment. Methods of determining whether a dosage resulted in the desired result would be evident to one of skill in the art. Administration of one or more antibodies can be continuous or intermittent, depending, for example, upon the recipient's physiological condition, whether the purpose of the administration is therapeutic or prophylactic, and other factors known to skilled practitioners. The administration of an antibody may be essentially continuous over a preselected period of time or may be in a series of spaced dose, e.g., either before, during, or after developing a target disease or disorder.

The present disclosure also provides kits for use in treating cancers. Such kits can include one or more containers comprising anti-neoantigen antibodies, e.g., any of those described herein and one or more immune checkpoint inhibitors. Such kits may also include one or more containers comprising effector cells comprising anti-neoantigen antibodies, e.g., any of those described herein and one or more immune checkpoint inhibitors.

In some embodiments, the kit can comprise instructions for use in accordance with any of the methods described herein. The included instructions can comprise a description of administration of the anti-neoantigen antibodies or effector cells comprising anti-neoantigen antibodies, and optionally the second therapeutic agent, to treat, delay the onset, or alleviate a target disease as those described herein. The kit may further comprise a description of selecting an individual suitable for treatment based on identifying whether that individual has the target disease, e.g., applying the diagnostic method as described herein. In still other embodiments, the instructions comprise a description of administering an antibody to an individual at risk of the target disease.

The instructions relating to the use of anti-neoantigen antibodies or effector cells comprising anti-neoantigen antibodies generally include information as to dosage, dosing schedule, and route of administration for the intended treatment. The containers may be unit doses, bulk packages (e.g., multi-dose packages) or sub-unit doses. Instructions supplied in the kits of the invention are typically written instructions on a label or package insert (e.g., a paper sheet included in the kit), but machine-readable instructions (e.g., instructions carried on a magnetic or optical storage disk) are also acceptable.

The label or package insert indicates that the composition is used for treating or alleviating a cancer. Instructions may be provided for practicing any of the methods described herein.

The kits of this invention are in suitable packaging. Suitable packaging includes, but is not limited to, vials, bottles, jars, flexible packaging (e.g., sealed Mylar or plastic bags), and the like. Also contemplated are packages for use in combination with a specific device, such as an inhaler, nasal administration device (e.g., an atomizer) or an infusion device such as a minipump. A kit may have a sterile access port (for example the container may be an intravenous solution bag or a vial having a stopper pierceable by a hypodermic injection needle). The container may also have a sterile access port (for example the container may be an intravenous solution bag or a vial having a stopper pierceable by a hypodermic injection needle). At least one active agent in the composition is an anti-neoantigen antibody or an effector cell comprising an anti-neoantigen antibody, such as those described herein.

Kits may optionally provide additional components such as buffers and interpretive information. Normally, the kit comprises a container and a label or package insert(s) on or associated with the container. In some embodiments, the invention provides articles of manufacture comprising contents of the kits described above.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, second edition (Sambrook, et al., 1989) Cold Spring Harbor Press; Oligonucleotide Synthesis (M. J. Gait, ed., 1984); Methods in Molecular Biology, Humana Press; Cell Biology: A Laboratory Notebook (J. E. Cellis, ed., 1998) Academic Press; Animal Cell Culture (R. I. Freshney, ed., 1987); Introduction to Cell and Tissue Culture (J. P. Mather and P. E. Roberts, 1998) Plenum Press; Cell and Tissue Culture: Laboratory Procedures (A. Doyle, J. B. Griffiths, and D. G. Newell, eds., 1993-8) J. Wiley and Sons; Methods in Enzymology (Academic Press, Inc.); Handbook of Experimental Immunology (D. M. Weir and C. C. Blackwell, eds.); Gene Transfer Vectors for Mammalian Cells (J. M. Miller and M. P. Calos, eds., 1987); Current Protocols in Molecular Biology (F. M. Ausubel, et al., eds., 1987); PCR: The Polymerase Chain Reaction, (Mullis, et al., eds., 1994); Current Protocols in Immunology (J. E. Coligan et al., eds., 1991); Short Protocols in Molecular Biology (Wiley and Sons, 1999); Immunobiology (C. A. Janeway and P. Travers, 1997); Antibodies (P. Finch, 1997); Antibodies: a practical approach (D. Catty., ed., IRL Press, 1988-1989); Monoclonal antibodies: a practical approach (P. Shepherd and C. Dean, eds., Oxford University Press, 2000); Using antibodies: a laboratory manual (E. Harlow and D. Lane (Cold Spring Harbor Laboratory Press, 1999); The Antibodies (M. Zanetti and J. D. Capra, eds., Harwood Academic Publishers, 1995).

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

### Example 1. Anti-Tumor Effects of Anti-Neoantigen Antibodies: Tumor Growth Retardation and Increased Survival in a Synergistic Model of Cancer

This study was designed to decrease tumor growth by treatment with multiple anti-neoantigen antibodies combined with PD1 inhibition. A dramatic reduction in tumor growth was observed. Despite tremendous excitement of checkpoint inhibitors in tumors, only a minority of patients have durable complete responses to checkpoint inhibition. The success of checkpoint inhibitors appears related to the immune response to neoantigens. The potential enhancement of checkpoint inhibitors was investigated by directing a cocktail of antibodies to multiple neoantigens simultaneously with PD1 inhibition.

Neoantigens were selected based on the potential rabbit humoral immunogenicity of a mutated epitope present at the tumor cell surface or secretary proteins, without assigning any weightage to the protein function. The epitopes selected were each 11 amino acids in length, and the mutated amino acid was located at or near the center of the epitope, as shown in Table 1 below.

TABLE 1
Selected Peptides Based on the Mutated
Regions of B16-F10 Melanoma Proteins
(normal and mutated amino acid is
bolded and underlined)

| | Normal | SEQ | Mutated | SEQ |
|---|---|---|---|---|
| Peptide | peptide | ID | peptide | ID |
| ID | sequence | NO: | sequence | NO: |
| c199l2 | GTTRAPSNPME | 1 | GTTRAQSNPME | 10 |
| fgfbpl | EKVRKRAKNAP | 2 | EKVRKGAKNAP | 11 |
| lama1 | NFRDFDTRREI | 3 | NFRDFNTRREI | 12 |
| usha2a | SVLSPLVKGQT | 4 | SVLSPPVKGQT | 13 |
| psg17 | RSRRETVYTNG | 5 | RSRREIVYTNG | 14 |
| psg25 | RETLHRNGSLW | 6 | RETLHSNGSLW | 15 |
| serpinc1 | GGRDDLYVSDA | 7 | GGRDDFYVSDA | 16 |
| tpo(ii) | KQMKALRDGDR | 8 | KQMKALRGGDR | 17 |
| ptgfrn | KLENWTDASRV | 9 | KLENWPDASRV | 18 |

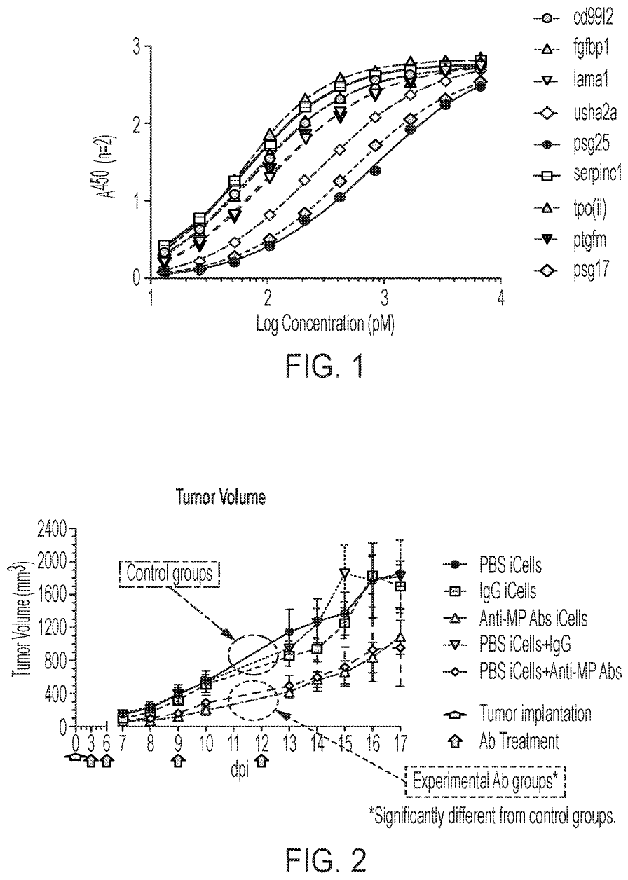

Rabbits were vaccinated with each of the 9 selected mutated epitopes of B16-F10 melanoma tumor proteins for generating high-affinity polyclonal antibodies to each peptide (FIG. 1). Rabbit polyclonal antibodies were affinity purified for each antigen with EC₅₀ values mostly in picomolar range, as shown in Table 2 below.

TABLE 2
EC₅₀ of Rabbit Antibodies against Selected
Mutated Peptides

| Antibody | EC₅₀ (M) |
|---|---|
| cd9912 | 7.64E−11 |
| fgfbp1 | 6.95E−11 |
| lama1 | 1.10E−10 |
| usha2a | 2.57E−10 |
| psg25 | 8.04E−10 |
| serpinc1 | 6.46E−11 |
| tpo(ii) | 6.14E−11 |
| ptgfrn | 9.39E−11 |
| psg17 | 4.92E−10 |

Experiment 1: Inoculation with Varying Numbers of B16-F10 Cells

To determine a number of B16-F10 cells to produce a tumor that grows to approximately 2000 mm³ size in 15-20 days, four groups of mice subcutaneously received 1×10⁴, 3×10⁴, 1×10⁵, or 3×10⁵ B16-F10 melanoma tumor cells suspended in PBS and tumor size was measured.

Tumor volumes increased more rapidly with higher numbers of tumor cells inoculated. Subcutaneous implantation of 300,000 B16-F10 cells produced very fast-growing tumors. By day 21 DPI, animals died or were euthanized because tumor size reached 2000 mm³ (data not shown). No unexpected weight changes were observed during this period. All treatment experiments used 300,000 tumor cells for implantation based on consistent rapid growth and lack of apparent side effects during tumor growth period. Also, during this short period of tumor growth, plasma antibodies to B16-F10 tumor lysates above background levels were not detectable (data not shown).

### Experiment 2: Determination of Effects of Antibody Cocktail on Tumor Growth and Animal Survival

All 9 polyclonal antibodies were mixed equally as a cocktail for treatment experiments. The tumor model used was the C57BL/J strain of mice implanted with 3×10⁵ B16-F10 melanoma tumor cells in the dorsal flank region. The mice were divided into five groups as treated as follows:

Gr 1. PBS Control: Mice received B16-F10 cells implantation in PBS.

Gr 2. Ig Control-1 Dose: Mice received B16-F10 cells implantation in 0.2 mg rabbit Ig (no further treatment).

Gr 3. Ab Cocktail-1 Dose: Mice received B16-F10 cells implantation in 0.2 mg cocktail of 9 anti-mutated peptides antibodies (no further treatment).

Gr 4. Ig Control-4 Doses: Mice received B16-F10 cells implantation in PBS+subcutaneous 0.2 mg rabbit Ig at the base of tumor on 3, 6, 9 and 12 days post implantation (DPI).

Gr 5. Ab Cocktail-1 Doses: Mice received B16-F10 cells implantation in PBS+subcutaneous 0.2 mg cocktail of 9 anti-mutated peptides antibodies at the base of tumor on 3, 6, 9 and 12 DPI.

The results are shown in FIG. 2. Between day 11 and day 12, the difference in tumor volume between the control groups and the experimental antibody groups became statistically significant. Two-way ANOVA showed significant tumor growth differences by the treatment (P<0.001) and time (P<0.001) factors, and their interaction (P=0.0105). The Tukey's multiple comparison test following two-way ANOVA determined significant tumor growth retardations in the groups of mice treated with a single dose (Ab Cocktail:1 Dose) or 4 doses (Ab Cocktail:4 Doses) of the 9-antibody cocktail in comparison to the mice of 3 control groups. Tumor growth for PBS (PBS Control:No Ab) and rabbit Ig control (Ig Control:1 Dose and Ig Control:4 Doses) groups of mice did not differ significantly.

The difference in survival was also statistically significant (FIG. 4). Median survival was 16 days for PBS, 17 days for Ig control-1 dose, 22 days for 9-antibody cocktail-1 dose, 16 days for Ig control-4 doses and 26 days for 9-antibody cocktail-4 doses group. Long-rank (Mantel-Cox) test of the data revealed a highly significant (P=0.0011) differences in the survival curves of the 4 groups of animals. Survival curves of antibody cocktail-treated groups were also significantly different from their respective Ig controls with a single dose (P=0.0136) or 4 doses (P=0.0141) of treatments. Survival curves for PBS control and Ig control groups did not differ significantly. The survival and hazard (Mantel-Haenszel) ratios between 1 dose Ig control and 9-antibody cocktail groups were 1.324 (95% CI 0.4269 to 4.104) and 0.1264 (95% CI 0.02444 to 0.6536), respectively. The survival and hazard ratios between 4 doses groups of Ig control and 9-antibody were 1.594 (95% CI 0.514-4.942) and 0.11 (95% CI 0.02148 to 4.942), respectively.

### Experiment 3: Determination of Effects of Antibody Cocktail and Anti-PD1 Antibody on Tumor Growth and Animal Survival

Experiment 2 was repeated to include the administration of an anti-PD1 antibody. A group of mice also received rat IgG2a isotype control of PD1i. The animals received subcutaneous implantation of 3×10⁵ B16-F10 melanoma tumor cells. The mice were divided into 7 groups and treated as follows:

Gr 1. PBS Control: Mice received B16-F10 cells implantation in PBS.

Gr 2. PBS+PD1i only: Mice received B16-F10 cells implantation in PBS+intraperitoneal 0.2 mg PD1i on 3, 6, 9 and 12 DPI.

Gr 3. Ig Control-1 Dose+PD1i: Mice received B16-F10 cells implantation in 0.2 mg rabbit Ig+intraperitoneal 0.2 mg PD1i on 3, 6, 9 and 12 DPI.

Gr 4. Ab Cocktail-1 Dose+PD1i: Mice received B16-F10 cells implantation in 0.2 mg cocktail of 9 anti-mutated peptides antibodies+intraperitoneal 0.2 mg PD1i on 3, 6, 9 and 12 DPI.

Gr 5. Ig Control-4 Doses+PD1i: Mice received B16-F10 cells implantation in PBS+subcutaneous 0.2 mg rabbit Ig at the base of tumor and intraperitoneal 0.2 mg PD1i, both treatments on 3, 6, 9 and 12 DPI.

Gr 6. Ab Cocktail-4 Doses+PD1i: Mice received B16-F10 cells implantation in PBS+subcutaneous 0.2 mg cocktail of 9 anti-mutated peptides antibodies at the base of tumor and intraperitoneal 0.2 mg PD1i, both treatments on 3, 6, 9 and 12 DPT.

Gr 7. PBS+PD1 Isotype Control: Mice received B16-F10 cells implantation in PBS+intraperitoneal 0.2 mg isotype control of PD1i antibody on 3, 6, 9 and 12 DPI.

As shown in FIG. 3, no treatment resulted in rapid growth of large tumors, and these untreated mice either died or were euthanized by day 20. The treatment with anti-PD1 antibody or its isotype control antibody alone did not affect tumor growth and survival in comparison to untreated mice. Furthermore, PD1 inhibition combined with normal rabbit IgG had no effect on tumor growth or survival (FIG. 3). However, the single treatment of tumor cells with the 9 antibody cocktail during implantation combined with PD1 inhibition significantly retarded tumor growth and increased survival (FIG. 3). The results were even more dramatic with the group of mice that were implanted with tumor cells in PBS and received 4 treatments of the 9 antibody cocktail in combination to PD1 inhibition. Two-way ANOVA of the data showed significant tumor growth differences by the treatment (P<0.001) and time (P<0.001) factors, and their interaction (P<0.001). Tukey's multiple comparison test following two-way ANOVA determined significant tumor growth retardations in the groups that received single and 4 doses of antibody cocktail plus PD1i compared to the PBS control (P<0.0001), PD1 alone control (P<0.0001), and respective Ig control groups with a single dose plus PD1i (P<0.0001) or 4 doses plus PD1i (P<0.0001) treatment.

Substantial enhancement of survival by the 1-dose and 4-dose antibody cocktail plus PD1i is shown as Kaplan Meier plots in FIG. 5. Furthermore, 50% of the mice treated with 4 doses of antibody cocktail and PD1i had complete and durable responses. These mice developed normal fur and survived the entire observation period for more than six months. In contrast, all the animals in the control groups died in less than 25 days of tumor implantation. The median survival were 16.5 days for PBS control, 18.5 days for PBS+PD1i, 18.5 days for Ig control-1 dose+PD1i, 22.5 days for Ab cocktail-1 dose+PD1i, 28 days for Ig control-4 doses+PD1i and 56 days for Ab cocktail-4 doses+PD1i group. Log-rank (Mantel-Cox) test of the data revealed a highly significant (P<0.0001) differences in the survival curves of the 6 groups of animals. The survival curves of the 1 and 4 dose antibody cocktail plus PD1i-treated groups were also significantly different from their respective Ig+PD1i control groups with a single dose (P=0.0006) or 4 doses (P=0.0006) of treatments. The survival curves of mice in PBS control, PD1i alone, isotype control of PD1i antibody (data not presented), and those in both Ig control+PD1i groups did not differ significantly. A high survival ratio of 1 dose 9-antibody cocktail+PD1i group 1.514 (95% CI 0.4881 to 4.693) was observed in comparison to 1 dose Ig control+PD1i group. Mantel-Haenszel test showed a lower hazard ratio of 1 dose 9-antibody cocktail+PD1i group (0.04314, 95% CI 0.00724 to 0.2571) compared to 1 dose Ig control+PD1i group. The survival and hazard ratios between 4 doses groups of Ig control and 9-antibody were 2.605 (95% CI 0.651 to 10.41) and 0.0464 (95% CI 0.00803 to 0.268), respectively.

Representative photos of mice 12 days following tumor inoculation are shown in FIG. 7. The dark fur had been shaved prior to tumor inoculation. Panel A is untreated, Panel B received PD1 inhibitor alone (PD1i), Panel C received a single dose of antibody cocktail+PD1i and has a small blush of pigmented tumor, and panel D received 4 doses of antibody cocktail+PD1i and showed no visible tumor. Large tumors are seen in PBS control and PD1i alone groups. The representing mouse that received a single dose of antibody cocktail plus PD1i displays a small blush of pigmented tumor. The representative mouse that received 4 doses of antibody cocktail plus PD1i shows no visible tumor.

### Experiment 4: Tumor Growth Following Re-Challenge

The surviving mice from Experiment 3 treated with 4 doses of antibody cocktail and PD1i were shaved and subcutaneously re-inoculated with 3×10⁵ B16-F10 melanoma tumor cells a second time, 6.5 months after the first tumor challenge. A group of never-treated mice were also implanted with 3×10⁵ B16-F10 melanoma tumor cells in the identical manner to serve as controls. Both groups of mice were then untreated, and their tumors were measured at different time intervals.

The animals of all the experiments were weighed 2 times/week and the tumor size were followed by measuring tumor volume (V=w²×½) every day starting from 7 days post-implantation using an electronic caliper. The survival time was calculated based on the death of an animal or euthanized following reaching to ≥2000 mm³ tumor volume. Following an IACUC approved protocol, the tumor-bearing animals were euthanized when tumor reached ≥2000 mm³ or when animals first exhibit signs of dehydration, difficulty walking, cachexia or other signs of physical distress.

Six-and-a-half months after a complete response to 4 doses of antibody cocktail and PD1i (FIG. 3), the mice were found to be persistently resistant to re-inoculation without any further treatment (data not shown). It is highly unlikely than any of the rabbit antibodies were present at the time of re-inoculation, so it seems that, following the initial treatment and complete response, these mice developed a robust anti-melanoma adaptive immune response. The untreated control mice had rapid tumor growth and no survival at 21 DPI.

### DISCUSSION

In this study, multiple tumor-specific proteins that were not overexpressed driver mutations were targeted. They are highly abundant in most, if not all, cancers. Rabbits vaccinated with peptides representing the mutated region of these tumor-specific proteins generated in high-affinity polyclonal antibodies. Individually, these antibodies had heterogeneous binding to B16-F10 cells. This reflects the variable and unpredictable expression of these proteins. However, combining the antibodies together as a cocktail resulted in strong homogeneous binding. These results support the idea that multiple antibodies to multiple tumor-specific neoantigens can overcome fundamental limitations associated with tumor heterogeneity. This idea is further supported by tumor inhibition experiments. A single dose of the cocktail of antibodies significantly inhibited tumor growth and prolonged survival of mice implanted with B16-F10 melanoma cells. Four doses substantially increased both tumor inhibition and survival.

PD1 inhibition of mouse melanoma has been reported in multiple studies. However, the range of treatment schedules and differing quantities of tumor cells inoculated have only demonstrated that the ability of anti-PD1 antibody to inhibit growth of B16-F10 melanoma cells is modest. A similar result was observed in the instant study: PD1 inhibition had minimal impact on tumor growth and survival. However, combining the cocktail of antibodies with an anti-PD1 antibody was found to result in substantial inhibition of tumor growth and prolongation of survival. The combination of four doses of the cocktail of antibodies with PD1 inhibition permanently prevented any melanoma growth in 50% of the mice.

Selection of mutated targets was based upon published NGS sequence data of B16-F10 melanoma cells. The targets selected were not driver mutations, and expression data was not used. The only criteria for selection were that the mutation was related to the cell surface by its presence in the membrane or as a secreted protein. For membrane proteins, selection was directed to those mutations not in the cytoplasmic or intramembrane portion of the membrane. Based on this information, short peptides representing the mutated section of the protein with the mutated amino acid in the center were designed. The set of peptides predicted to be immunogenic in the rabbit were then synthesized and used as vaccines. All of the selected peptides produced high titers of antibodies. Affinity enrichment yielded 4 to 6 mg of high-affinity polyclonal antibodies. The majority of the antibodies bound B16-F10 cells and, when combined together, produced remarkable tumor inhibition of a relatively aggressive tumor.

To assess the possible applicability of this approach for treating cancer patients the single non-synonymous mutations of four breast cancer patients from published sequence data were analyzed to determine how many mutations were membrane-associated. The total number of gene mutations in these patients ranged from 15 to 221. The cell location of each identified gene mutation was determined using the human protein atlas project or the human gene database through GeneCards. It was determined that 27%-46% of all the non-synonymous mutations reported in these breast cancer patients were proteins found on the plasma membrane. Therefore, cancer cell surface mutations in breast cancer patients are not uncommon and are available for targeting with antibodies.

No adverse events were observed in the treated mice. Antibodies, even from different species, are not inherently toxic. The strategy of using tumor-specific mutations may reduce cross-binding to normal proteins. Without wishing to be bound by theory, it is thought that using an antibody cocktail which diminishes the dose of each antibody required for efficacy may further diminish possible normal cell toxicity to any individual antibody. A normal cell that expresses a cross-reactive antigen would be exposed to a relatively low dose of that individual antibody. The likelihood of normal cells binding to additional antibodies from the cocktail becomes increasingly remote as the number of different antibodies in the cocktail increases.

Cell surface mutations that are not overexpressed or involved in maintaining the malignant phenotype have been largely ignored as potential targets for therapy. It appears that such mutations are in abundance. It also appears that there is considerable variation between patients in which proteins are mutated. These types of mutations would normally be considered of low value from the perspective of developing single pharmaceutical reagents that can be used for many patients.

### Example 2. Antibodies Targeting Tumor-specific Mutations Redirect Immune Cells to Inhibit Tumor Growth and Increase Survival

The purpose of this study was to determine whether effector cells could be “armed” with the anti-neoantigen antibodies in vitro and then systemically administered to mice as a cell therapy.

Antibodies were generated as described in Example 1. Rabbits vaccinated with the selected mutated peptide-KLH conjugates successfully generated high titer sera against each mutated peptide. Affinity purification of sera against individual mutated peptides yielded highly purified polyclonal antibodies. ELISA titration of serially (2-fold) diluted purified antibody samples against related mutated peptides revealed high titer (≥1:500,000) values. The calculations of ECso value of each antibody sample provided an affinity estimate against their targets. The ECso values of the rabbit polyclonal antibodies presented in Table 3 show picomolar level affinities against related mutated peptide targets.

TABLE 3
EC50 of Rabbit Antibodies against
Selected Mutated Peptides

| | | | SEQ | Abs |
|---|---|---|---|---|
| Peptide | | Mutated | ID | EC50 |
| ID | Mutation | peptide | NO: | (M) |
| Cd99l2 | P86Q | GTTRAQSNPME | 10 | 5.41E−11 |
| Fgfbp1 | R26G | EKVRKGAKNAP | 11 | 4.30E−11 |
| Lama1 | D656N | NFRDFNTRREI | 12 | 7.91E−11 |
| Ush2a | L4514P | SVLSPPVKGQT | 13 | 5.84E−11 |
| Psg17 | T340I | RSRREIVYTNG | 14 | 9.46E−11 |
| Psg25 | R1025 | RETLHSNGSLW | 15 | 5.97E−11 |
| Serpinc1 | L395F | GGRDDFYVSDA | 16 | 4.91E−11 |
| Tpo(II) | D656G | KQMKALRGGDR | 17 | 4.82E−11 |
| Ptgfrn | T620P | KLENWPDASRV | 18 | 2.26E−10 |

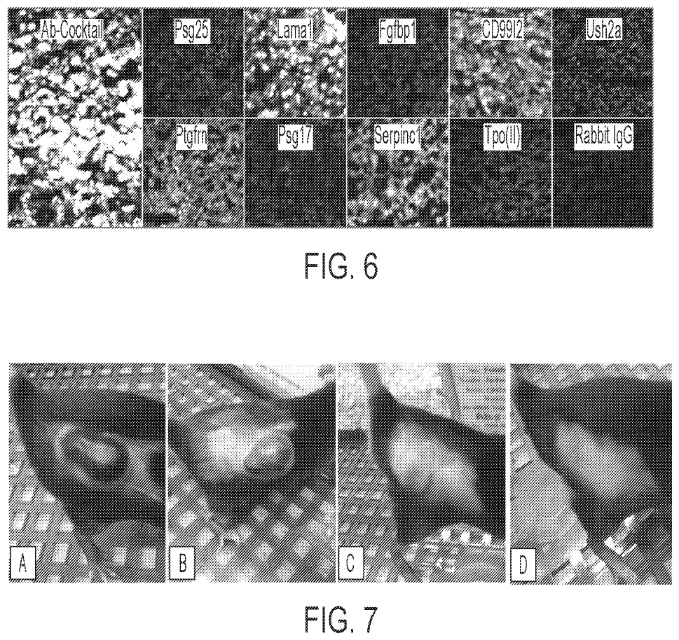

Fluorescence microscopic binding analysis of individual polyclonal antibodies to mouse B16-F10 tumor sections demonstrated positive binding by all the purified antibody samples (FIG. 6). The results demonstrate a variation in the staining intensities of mouse B16-F10 tumor sections by individual antibodies. However, tumor binding with a cocktail of all 9 antibodies produced uniformly high intensity-stained tumor sections. Antibodies generated against the mutated peptides Lamal, Ptgfrn, CD9912 and Serpinc1 displayed higher tumor tissue binding intensities in comparison to other antibodies. These antibodies were designated as 4 high-binding antibodies for the animal studies.

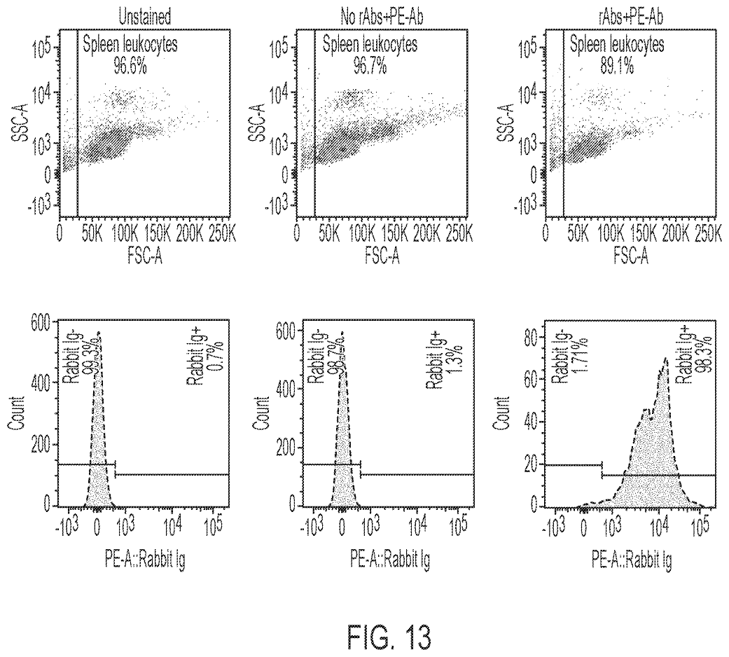

FIG. 13 presents the binding of the 9-antibody cocktail to spleen leukocytes evaluated by flow cytometry. The gating was performed to include most of the live spleen cells. The results show that ~98% of leukocytes bind to the rabbit antibodies in the cocktail (lower right panel). It was also observed that donkey antibodies binding to mouse spleen leukocytes was negligible (lower middle panel). Furthermore, there was no difference in the mouse leukocyte binding to rabbit antibody whether the samples were blocked by donkey serum samples before or after rabbit antibody incubation step (data not shown). These controls ruled out any interference with the blocker and secondary antibody.

### Experiment 1: Combined Treatment of Armed Effector Cells and PD1 Inhibitor

This experiment was conducted to determine the effect of combined treatment with mouse spleen effector cells armed with a cocktail of rabbit polyclonal antibodies against the 9 tumor-specific mutated proteins (“Ab cocktail”) and anti-mouse PD1 inhibitor antibody (PD1i) on the B16-F10 melanoma tumor growth and survival. All the mice in this experiment received subcutaneous implantation of 3×10⁵ B16-F10 melanoma tumor cells. The mice were divided into 4 groups and treated as follows:
Gr 1. PBS Control: Mice received B16-F10 cells implantation in PBS.
Gr 2. PBS+PD1i only: Mice received B16-F10 cells implantation in PBS+intraperitoneal 0.2 mg PD1i on 3, 6, 8, 10, and 13 days post-implantation (DPI).
Gr 3. EC+PD1i: Mice received B16-F10 cells implantation in PBS+subcutaneous injection of 1×10⁷ spleen leukocytes (effector cells, EC) at the tumor base+intraperitoneal 0.2 mg PD1i, both on 3, 6, 8, 10, and 13 DPI.
Gr 4. EC-armed with Ab Cocktail+PD1i: Mice received B16-F10 cells implantation in PBS+subcutaneous injection of 1×10⁷ spleen leukocytes-armed with 9-Ab cocktail at the tumor base+intraperitoneal 0.2 mg PD1i, both on 3, 6, 8, 10, and 13 DPI.

The results of combined treatment on tumor growth at different time intervals post-implantation are presented in FIG. 10. The two-way ANOVA of the data showed significant tumor growth differences by the treatment (P=0.0252) and time (P<0.001) factors. The treatment with PD1i alone or PD1i in combination with effector cells did not affect the tumor growth significantly in comparison to the PBS control group. However, a combined treatment of effector cells armed with a cocktail of 9 antibodies and PD1i (“EC-armed with Ab cocktail+PD1i”) significantly retarded the tumor growth in comparison to PBS control groups. Tumor growth in this group of mice were reduced by −39% (7 days post-implantation, DPI), −34% (8 DPI), −42% (9 DPI) −47% (10 DPI) and −49% (13 DPI) in comparison to PBS control. The Tukey's multiple comparison test following two-way ANOVA determined significant tumor growth retardations in this group of mice treated with the EC-armed with Ab cocktail+PD1i in comparison to both the PBS control and PD1i alone groups at 13 DPI.

The Kaplan-Meier plot in FIG. 8 presents mice survival data. A log-rank trend test of the data revealed a significant (P=0.033) trend in the survival curves. The treatment of B16-F10 melanoma tumor bearing mice with a combined treatment of effector cells armed with a cocktail of antibodies against 9 selected mutated proteins and PD1i significantly increased the survival of mice in comparison to the mice of control groups. The median survival times were 16.5 days for PBS, 18.5 days for PD1i alone, 20 days for effector cells+PD1i, and 25 days for effector cells-armed with 9-antibody cocktail groups. The survival ratio of effector cells armed with antibodies and PBS control was 1.212 with a 95% confidence of interval (CI) of the ratio ranging from 0.3699 to 3.972. The hazard ratio (log-rank) of these two groups was 0.5591 (95% CI, 0.1675 to 1.866).

Experiment 2: Combined Treatment of Effector Cells Armed with Four- or Nine-Antibody Cocktail and PD1 Inhibitor (PD1i)

This experiment was conducted to determine the effectiveness of combined treatment with spleen leukocytes-armed with 4 high tumor-binding anti-mutated antibodies and PD1i. The experiment also examined a shorter time interval of injection of effector cells (from every 2-3 days to every 1 day) on B16-F10 melanoma tumor growth and survival. The animals received subcutaneous implantation of 3×10⁵ B16-F10 melanoma tumor cells. The mice were divided into 4 groups and treated as follows:
Gr 1. PBS Control: Mice received B16-F10 cells implantation in PBS.
Gr 2. PBS+PD1i only: Mice received B16-F10 cells implantation in PBS+intraperitoneal 0.2 mg PD1i on 3, 5, 7, 10 and 12 DPI.
Gr 3. EC-armed with 9-Ab Cocktail+PD1i: Mice received B16-F10 cells implantation in PBS+subcutaneous injection of 1×10⁷ spleen leukocytes—armed with 9-Ab Cocktail at the tumor base on 3, 4, 5, 6, and 7 DPI+intraperitoneal 0.2 mg PD1i on 3, 5, 7, 10, and 12 DPI.
Gr 4. EC-armed with 4HB-Ab Cocktail+PD1i: Mice received B16-F10 cells implantation in PBS+subcutaneous injection of 1×10⁷ spleen leukocytes armed with the cocktail of 4 high-binding (HB) antibodies at the tumor base on 3, 4, 5, 6, and 7 DPI+intraperitoneal 0.2 mg PD1i on 3, 5, 7, 10, and 12 DPI.

The results of combined treatment of effector cells-armed with 4- or 9-antibody cocktail and PD1 inhibitor on tumor growth in mice implanted with 3×10⁵ B16-F10 cells are presented in FIG. 12. In this experiment, mice received daily treatments of effector cells armed with a cocktail of 4 high binding antibodies or all 9 antibodies for 5 days starting from 3 days post-implantation (DPI). The results show a significant retardation in tumor growth in both groups treated with effector cells armed with antibodies and PD1i.

The two-way ANOVA of the data showed significant tumor growth differences by the treatment (P<0.0001) and time (P=0.0013) factors. The treatment with PD1i alone did not produce any significant effects in comparison to PBS control. However, the combined treatment of effector cells armed with 9 antibodies and PD1i (“EC-armed with 9-Ab Cocktail+PD1i”) daily for 5 days retarded the tumor growth by −44%, −63%, −67%, −68% and −60% at 7, 10, 11, 12, and 13 DPI, respectively, in comparison to the PBS control group. Further, high degrees of tumor growth retardations were observed in mice co-treated with effector cells armed with the cocktail of 4 high-binding antibodies (“EC-armed with 4HB-Ab Cocktail”) and PD1i (−64% at 7 DPI, −84% at 10 DPI, −87% at 11 and 12 DPI, −85% at 13 DPI) in comparison to the PBS control. The Tukey's multiple comparison test following two-way ANOVA determined highly significant tumor growth retardations in the mice of 4HB-Ab cocktail group in comparison to both the PBS control (11 DPI, P=0.0222; 12 DPI, P=0.0025; 13 DPI, P=0.0127) and the PD1i alone (11 DPI, P=0.0089; 12 DPI, P=0.0006; 13 DPI, P=0.0069) groups.

The Kaplan Meier plot in FIG. 14 presents survival data following the combined treatment with effector cells armed with a cocktail of the 4 high binding or all 9 antibodies and PD1i. A log-rank (Mantel Cox) test of the data revealed highly significant (P<0.0007) differences in the survival curves of the 4 groups of animals. The survival curves of mice co-treated with effector cells armed with the 4- or 9-antibody cocktail plus PD1i were significantly different (P=0.0024) from the PBS control group. The combined treatment of B16-F10 melanoma tumor bearing mice with the cocktail of 4 high tumor-binding antibodies against selected mutated proteins and PD1i was most effective and produced the maximum survival curve for 31 days. All of the animals in the PBS control group died by 19 days. The median survival durations were 17 days for PBS, 19.5 days for PD1i alone, 22.5 days for effector cells armed with all 9 antibodies+PD1i, and 31 days for effector cells-armed with 4 high binding antibody cocktail groups. The median survival ratio of effector cells armed with 9 antibodies group and PBS control was 1.324 with the 95% CI ranging from 0.404 to 4.337. The log-rank hazard ratio of these two groups was 0.249 (95% CI, 0.06113 to 1.014).

To assess the feasibility of this approach for treating human cancer patients, the number of non-synonymous mutations expressed on the extracellular surface of membrane-associated or secreted proteins was determined (Table 4).

TABLE 4
Number of Detected Missense Mutations in Extracellular
Domain of Cell Membrane Proteins or Secreted Proteins

| | | | # Mutations |
|---|---|---|---|
| | | # Mutations ECD Cell | Secreted |
| Cancer Type | Sample ID | Membrane Protein | Protein |
| Breast | BC1 | 8 | 8 |
| Breast | BC2 | 24 | 26 |
| Breast | BC3 | 3 | 1 |
| Breast | BC4 | 8 | 5 |
| Breast | BC5 | 9 | 3 |
| Breast Cancer Average | | 10.4 | 8.6 |
| Neuroblastoma | BIO-275-08 | 18 | 7 |
| Neuroblastoma | BIO-284-08 | 14 | 9 |
| Neuroblastoma | BIO-295-08 | 1 | 2 |
| Neuroblastoma | BIO-332-08 | 5 | 2 |
| Neuroblastoma | BIO-296-08 | 2 | 0 |
| Neuroblastoma Average | | 8 | 4 |
| Melanoma | CR3655 | 16 | 14 |
| Melanoma | CR4880 | 69 | 52 |
| Melanoma | CR9306 | 179 | 131 |
| Melanoma | SR1494 | 130 | 120 |
| Melanoma | SR2056 | 21 | 11 |

| | | |
|---|---|---|
| Melanoma Average | 83 | 65.6 |

The total number of gene mutations in the breast cancer patients ranged from 15 to 252 and in the neuroblastoma patients, 17-128. The UniProt protein database was also used, and it was found that breast cancers had an average of 10 nonsynonymous mutations (range, 3 to 24) located on the extracellular region of cell membrane proteins and, on average, 9 nonsynonymous mutations (range, 1 to 26) expressed on secreted proteins.

In the neuroblastoma tumors, there were an average of 8 mutations (range, 1 to 18) per patient on the extracellular region of cell membrane proteins and an average of 5 mutations (range, 2 to 9) expressed on secreted proteins.

In malignant melanomas, an average of 83 mutations (range, 16 to 179) per tumor on the extracellular region of cell membrane proteins and an average of 66 mutations (range, 11 to 131) per tumor expressed on secreted proteins.

Therefore, there are a number of non-synonymous mutations expressed on the extracellular surface of membrane-associated or secreted proteins in human cancers.

### DISCUSSION

As described in Example 1, when multiple antibodies targeting 9 different tumor-specific mutations were combined and delivered as a cocktail not bound to effector cells there was remarkable inhibition of tumor. Further experiments were undertaken to examine whether the antibody cocktail may be used to redirect immune effector cells, a method that would require significantly lower levels of antibody.

A set of nine polyclonal antibodies were generated by vaccinating rabbits with the selected mutated peptides. Each of the 9 affinity enriched antibodies showed variable binding intensity to histologic sections of B16-F10 tumor harvested from a mouse (Table 2). When all 9 antibodies were combined, tumor cell binding was uniform and intense across the cell population.

Unsorted splenic immune effector cells were harvested from syngeneic mice. These effector cells were incubated with the cocktail of 9 antibodies and injected into mice previously inoculated with B16-F10 cells. Similar to the experiments described in Example 1, the cocktail of 9 antibodies loaded on effector cells combined with PD1 inhibitor caused tumor inhibition and increased survival of mice. However, these desirable outcomes were achieved at a dramatically lower dose of antibodies relative to those used in Example 1. Each dose of armed effector cells included no more than 1 microgram of the cocktail of antibodies. It was unexpected that a total dose of antibodies less than 5 micrograms (from 5 separate injections) is capable of inhibiting the tumor and prolonging survival.

The small amount of antibody required to saturate Fc receptors makes the possibility of clinical studies more feasible. For example, based on the types of effector cells in human peripheral blood and the molar mass of antibodies the estimated amount of antibodies needed to arm 1×10⁹ effector cells is about 8-75 micrograms^{16,17}. There are a variety of strategies to obtain such small quantities of antibodies including affinity enrichment following vaccination of a small animal as was done here. It was also found that arming effector cells with a set of higher binding antibodies increased the effectiveness of inhibiting tumor cells.

Two experimental modifications were evaluated to improve tumor inhibition outcomes. The first was to change the frequency of treatment from every 2-3 days to every day to account for short duration of effector cell activity. In this study, binding to splenic effector cells was assessed only to three hours. However, the duration of antibodies binding to effector cells via Fc receptors is transient and likely lasts less than 12 hours^{18,19}. With a short-time interval for armed cells to be bioactive, treatment every three days would allow time for this highly aggressive melanoma to grow between treatments. Indeed, the same total number of effector cells and antibody dose given daily instead of every 2-3 days as shown to increase tumor inhibition and increase survival.

The second experimental modification was designed to increase bioactivity of the armed effector cells to the target melanoma cells. With free antibodies, individual antibodies bind the B16-F10 melanoma cells according to target density. However, when the 9 antibodies are restricted via Fc binding on effector cells, saturation of tumor targets at the effector and tumor cell interface may be limited. The tumor presents variable target density at its interface. However, the effector cell presents antibodies bound to Fc receptors that are in equal distribution on the effector cell surface. With a mix of 9 antibodies that bind different tumor targets, there may be insufficient antibodies at the interface to fully occupy higher density tumor binding sites. To test this, a set of effector cells was armed with the 4 antibodies (out of the 9 antibodies) that showed the most intense binding to B16-F10 cells. Arming effector cells with these 4 antibodies resulted in increased tumor growth inhibition and increased survival.

A fundamental challenge to successfully treat cancer is tumor heterogeneity. Herceptin illustrates how profoundly heterogeneity limits the ability to cure patients. This is a prototype blockbuster drug widely used to treat breast cancer when HER2 is overexpressed. However, expression of HER2 is variable across the population of cancer cells. When binding of Herceptin falls below a certain threshold, it will not mediate cell death. This population of cells will continue to grow. Although Herceptin caused delay to progression of about 3 months in patients with stage IV breast cancer none of these patients were cured²⁰. Cell-based therapies such as CAR-T cell therapy must overcome the same challenge faced with monoclonal antibody treatments. The tumor population will have variable expression of any single tumor-related target. When that expression falls below the threshold necessary for CAR-T cells to bind and mediate cell death, such cells will invariably continue to grow. This limitation has considerably reduced the clinical application of CAR-T cell therapy^{5,21}. Indeed, the first FDA approved CAR-T cell treatment did not target tumor cells specifically but targeted all cells bearing normal CD19²². Adverse events with this approach are not simply off-target effects, they are the result of intentional ablation of normal cells.

Arming effector cells with a multiplicity of tumor-specific antibodies offers a strategy to overcome the challenge of heterogeneity. Individually, each of the 9 antibodies had variable binding to B16-F10 cells representing heterogeneous expression on the B16-F10 cells. When combined there was strong homogeneous binding.

In this study, tumor-specific mutations were targeted. Targeting tumor-specific mutations can mitigate non-tumor tissue destruction that occurs, for example, with CD19 targeted T cell therapy. Serial treatments to address tumor regrowth and resistance may be possible by resequencing the tumor and identifying additional tumor-specific mutations as targets. Neutral tumor-specific proteins that were not known to be driver mutations were also targeted. These were random mutations that are highly tumor-specific. These types of mutations appear to be common in multiple types of human cancers. Sequence data from human specimens of melanoma, breast, and neuroblastoma showed that nonsynonymous cell surface mutations were present and, in many cases, abundant. This preliminary analysis of a variety of solid human tumors demonstrates that the approach described herein may be translatable to clinical studies.

Controlling the final amount and type of cellular reagents with tumor-infiltrating lymphocytes (TIL) or CAR-T therapy is difficult. A very high single dose is given which requires preliminary reduction of existing T cells. Such a high dose puts a patient at risk for a variety of adverse events such as cytokine release syndrome and death²³. As a living reagent, a method to reliably and selectively shut down therapeutic T cells is not available. Treating patients with armed effector cells as described herein allows the possibility of exquisite control of dose and duration of bioactive cell reagents. Delivery is relatively simple and effector cells are in abundance in peripheral blood. The short duration of antibodies binding to effector cells provides a built-in safety feature and stopping treatments should rapidly cease ongoing bioactivity of the effector cells. Serial treatments were accomplished without evidence of adverse events in the mice.

This study used unsorted splenic cells as effector cells. Previous studies have demonstrated that multiple types of effector cells derived from spleen, peripheral blood, marrow, and the peritoneum can mediate cytotoxicity when armed with immune sera^{27,28}. Using only one immune effector cell type (e.g., T cell therapy), does not take full advantage of multiple mechanisms of tumor cell destruction offered by different immune effector cells²⁹. The ability to grow T cells has factored heavily in the choice of cell type for most trials. However, as described herein, the complexity and expense related to growing T cells ex vivo may be avoided. Multiple types of effector cells are readily available from peripheral blood and do not require ex vivo growth expansion. Further, genetic modification to introduce a binding element, as is done with CAR-T cells is unnecessary, since effector cells already have a good handle for antibodies.

Thus, ex vivo arming a mixed population of immune effector cells with antibodies targeting multiple tumor-specific mutated proteins in conjunction with PD1 inhibition was shown to delay tumor growth and prolong survival in mice inoculated with an aggressive melanoma.

### Materials and Methods (Examples 1 and 2)

### Reagents and Cell Line

B16-F10 melanoma tumor cells were procured from American Type Culture Collection (ATCC, Manassas, VA). Normal rabbit IgG was supplied by Sino Biological, Inc. (Wayne, PA). Alexa Fluor 568-conjugated goat anti-rabbit antibody was procured from Life Technologies (Carlsbad, CA). Dulbecco's Modified Eagle's Medium (DMEM) and Trypsin-EDTA solution were purchased from ATCC. All other reagents used in this study were of molecular or high purity grade. Rat anti-mouse PD1 (CD279) antibody and rat IgG2a isotype control of anti-PD1 antibody were purchased from Bio X cell (West Lebanon, NH).

### Selection of Tumor-Specific Mutated Proteins and Antibody Production

Confirmed sequence data of B16-F10 mouse melanoma tumor cell line⁷ was analyzed, and multiple cell-surface-related mutated proteins with a single amino acid substitution were selected. The 11-mer peptides representing the mutated region of the mutated proteins were designed, keeping the mutated amino acid residue in or near the center (Table 1). The B cell response and immunogenicity predictions were estimated by comparing the homologies of the selected epitopes in mouse with rabbit.

Peptide synthesis and rabbit vaccination for antibody production were done by GenScript (Piscataway, NJ). Briefly, a cysteine residue that was automatically added at C-terminus when synthesizing the peptides was used for conjugating the peptides to Keyhole Limpet Hemocyanin (KLH) protein. Following rabbit vaccination with peptide-KLH conjugates, sera were collected, and affinity-purified against an individual mutated peptide. The affinity estimations (EC₅₀) of individual purified polyclonal antibodies for their respective mutated peptides were done by tittering the samples using ELISA⁸. Individual mutated peptides were immobilized in flat-bottom clear MaxiSorp 96-well plates (Nunc, Rochester, New York) and after blocking the wells with 1% BSA in PBS, the plates were washed 3 times with PBS. Serially (2-fold) diluted purified antibodies were incubated with the immobilized mutated peptides. HRP-conjugated anti-rabbit IgG antibodies and HRP substrate 3,3′,5,5′-tetramethylbenzidine (TMB) reagent (GenScript) were used for determining the antibody binding to the mutated peptides. Following the color development, wells were read for their absorbance at 450 nm using a Synergy HT plate reader (BioTek Instruments, Inc. Winooski, VT).

### Binding Analysis of Rabbit Polyclonal Antibodies by Immunofluorescence Microscopy

The frozen cut sections of mouse B16-F10 tumor tissue mounted on glass slides were fixed in 3% Paraformaldehyde (Electron Microscopy Sciences, Hatfield, PA) and washed with PBS three times (3 times, three minutes each) for immunofluorescence microscopy. The slides were blocked with Image-iT™ FX Signal Enhancer (Thermo Fisher Scientific, Waltham, MA) for 30 minutes at room temperature in a humidified chamber. The tissue sections were rinsed again 3 times in PBS and incubated with single or pooled rabbit polyclonal antibodies and normal rabbit IgG for 1 hour at room temperature. Slides were rinsed 3 times in PBS and incubated with Alexa Fluor 568-conjugated goat anti-rabbit IgG (H+L) antibody (Invitrogen, CA) for 1 hour at room temperature. The slides were rinsed in PBS, cover-slipped with Dako Fluorescent Mounting Medium (Agilent, Santa Clara, CA) and analyzed at excitation 579 nm and emission 603 nm and excitation 359 nm and emission 461 nm using a Nikon TE2000-U inverted fluorescence microscope (Nikon Corp., Kangawa, Japan).

### Mouse Spleen Leukocyte Preparation

Mice were euthanized by carbon dioxide inhalation based on the approved University of Vermont Institutional Animal Care and Use Committee (UVM IACUC) protocol. Spleens were collected under sterilized condition. Spleens were trimmed to remove adhering fat tissue and cut into small pieces under the hood. Spleen pieces were kept between two 150 μm sterilized nylon meshes in a petri dish with Dulbecco's phosphate-buffered saline (DPBS) and gently pressed with the plunger end of a syringe. The disaggregated spleen pieces and liberated cells were collected and passed through a 70 μm cell strainer to produce a single cell suspension. The cells were rinsed two times in DPBS and red blood cells were removed by incubating the cells with Gibco ACK Lysing Buffer (Life Technologies Corp., Grand Island, NY) at 37° C. for 3 minutes. The cells were counted with viability determination using trypan blue. Spleen leukocytes from multiple mice were pooled for the study.

Arming of Spleen Effectors Cells with Antibodies

Spleen leukocyte samples were mixed with the antibody cocktail of rabbit anti-mutated peptide antibodies (1×10⁸ cells+100 μg antibodies/mL PBS) and incubated for 30 minutes in ice. The samples were centrifuged, and the leukocyte pellet was suspended in cold PBS (1×10⁸ cells/mL). The cells were kept on ice and used immediately.

### ELISA for Binding of Tumor-Bearing Mouse Plasma Antibodies to B16-F10 Tumor Lysate

The lysate tumor preparation and lysate ELISA were performed using methods known in the art. Briefly, the lysate of B16-F10 tumors grown in untreated mice were prepared by freeze/thaw cycles and ultrasonication. Then, 50 μL cell lysate (1 mg/mL protein) was immobilized and blocked with 1% casein-TBS (Thermo Fisher). Then, diluted (100, 200 and 400×) plasma of normal and tumor-bearing mice was added and the plates were incubated for 2 hours at room temperature. The wells were washed and incubated with cross-absorbed goat anti-mouse IgG (H+L)-horseradish peroxidase (HRP) conjugate (Life Technologies, Carlsbad, CA). The lysate-bound plasma antibodies were then detected with TMB soluble substrate (Calbiochem, Billerica, MA).

### Flow Cytometric Analysis of Spleen Leukocyte-Binding to Anti-Mutated Peptide Antibodies

Flow cytometry was used to analyze the binding of rabbit antibodies to spleen leukocytes using a standard protocol described previously⁹. Briefly, the cells in PBS containing 1% BSA were blocked with 10% (v/v) donkey serum (Southern Biotech, Birmingham, AL) before or after incubation with rabbit polyclonal anti-mutated peptide antibodies (1×10⁷ cells/1.1 g each Ab/100 μL) raised against mutated peptides. The cell-bound rabbit Ig were detected with Phycoerythrin-conjugated donkey anti-rabbit Ig (H+L) antibody (Biolegend, San Diego, CA). All the incubation steps were for 30 minutes on ice followed by three (3 min each) washings with PBS containing 1% BSA. The stained cells were fixed in 2% paraformaldehyde and following PBS-washes the samples were run on BD LSRII (BD Biosciences, San Jose, CA) flow cytometer equipped with BD FACSDiva™ version 8 software for data acquisition. The data were analyzed using FlowJo™ v10.1 software (FlowJo, LLC, Ashland, OR) following required gating with the help of proper controls.

### Animal Tumor Model

A well-established syngeneic tumor model representing a spontaneous C57BL/6-derived B16-F10 melanoma tumor was used as a model system for this study. This is a non-immunogenic very aggressive mouse tumor. The Jackson Laboratory (Bar Harbor, ME) supplied 6-7 weeks old (date of birth±3 days) female mice of C57BL/6J strain. Mice were kept in animal housing maintained at the standard conditions with 12-h light/dark cycles and provided with food pellets and water ad libitum. Following one week of acclimatization, mice were ear-punched for numbering and had their right flank body region shaved. B16-F10 melanoma tumor cells were cultured to ~70% confluency in Dulbecco's modified Eagle medium containing 10% (v/v) fetal bovine serum according to the suggestions of the supplier. The cells were tested for mycoplasma infection to confirm negative prior to implantation. The washed cells were suspended in PBS and subcutaneously injected at the shaved right flank of the mice. All the animal procedures (Protocol #18-002) used in the present study were approved by the UVM IACUC.

### Tumor Growth and Survival

The growth of B16-F10 tumors was followed by measuring tumor volume (v=(w²×L)/2) every day starting from 7 days post-implantation (DPI) using an electronic caliper. The survival time was calculated based on the animal death or euthanasia following tumor volume reaching to ≥2000 mm³. According to the approved TACUC protocol, the tumor-bearing animals were required to be euthanized when tumor volume reached ≥2000 mm³ or when animals exhibited signs of dehydration, difficulty walking, cachexia or other signs of physical distress, whichever came first. The body weights were recorded twice a week for monitoring general health of the tumor-bearing mice.

### Statistical Analyses

One-way and Two-way ANOVAs followed by Tukey's multiple comparison test were used to evaluate the tumor growth differences in the animals of different treatment groups. The survival data were presented as Kaplan Meier plot. The significance of differences among survival curves of treatment groups were analyzed using Log-rank (Mantel-Cox) test and median survival values. The confidence intervals (CT) for means of median survival and hazard (Mantel-Haenszel) ratios at 95% confidence level were also estimated. GraphPad (San Diego, CA) Prism software was used for some of the analyses.

### Mutation Analysis in Cancer Patients

The data used for the analysis of somatic mutations of cancer patients came from three sources. The five breast cancer patients and melanoma whole exome sequence data and mutation identification were downloaded from the publications by Wan et al. 2011 and Snyder et al. 2014^{10,11}. The whole exome sequencing of the five neuroblastoma patients were obtained from patients treated at Helen Devos Children's Hospital enrolled on The Signature Study: Molecular Analysis of Pediatric Tumors with Establishment of Tumor Models in an Exploratory Biology Study: NMTRC OOB. Sequencing was performed at the Translational Genomics Research Institute. DNA extraction by Qiagen AllPrep and libraries generated with KAPA Hyper (Illumina) and captured with a supplemented IDT xGen exome kit. Libraries were then clustered on flow cell and sequenced using the Novaseq 6000 (Illumina). Somatic point mutations and indels were detected by VarScan2.3.9 (VarScan2.3.9 somatic -min-coverage 20 --somatic-p-value 0.001 -min-var-freq 0.05 -min-avg-qual 30) and later annotated with SnpEff software^{12,13}. Variant Effect Predictor (VEP) was used to convert the genetic variants on genes or transcripts to protein mutants on the protein level¹⁴. Only missense variants were considered for further analysis. The subcellular locations and extracellular location for all the mutated proteins with were completed with UniProt database¹⁵. The cell membrane protein mutants or secreted protein mutants were selected. For cell membrane protein mutants the only the mutated sites included in the analysis are those within extracellular regions of the protein.

### REFERENCES

1. Topalian S L, Muul L M, Solomon D, Rosenberg S A. Expansion of human tumor infiltrating lymphocytes for use in immunotherapy trials. J Immunol Methods 1987; 102:127-41.
2. Rosenberg S A, Packard B S, Aebersold P M, et al. Use of tumor-infiltrating lymphocytes and interleukin-2 in the immunotherapy of patients with metastatic melanoma. A preliminary report. N Engl J Med 1988; 319:1676-80.
3. Rosenberg S A, Dudley M E. Cancer regression in patients with metastatic melanoma after the transfer of autologous antitumor lymphocytes. Proc Natl Acad Sci USA 2004; 101 Suppl 2:14639-45.
4. Yannelli J R, Hyatt C, McConnell S, et al. Growth of tumor-infiltrating lymphocytes from human solid cancers: summary of a 5-year experience. International journal of cancer 1996; 65:413-21.
5. Minutolo N G, Hollander E E, Powell D J, Jr. The Emergence of Universal Immune Receptor T Cell Therapy for Cancer. Front Oncol 2019; 9:176.
6. Porter D L, Levine B L, Kalos M, Bagg A, June C H. Chimeric antigen receptor-modified T cells in chronic lymphoid leukemia. N Engl J Med 2011; 365:725-33.
7. Castle J C, Kreiter S, Diekmann J, et al. Exploiting the mutanome for tumor vaccination. Cancer Res 2012; 72:1081-91.
8. Shukla G S, Sun Y J, Pero S C, Sholler G S, Krag D N. Immunization with tumor neoantigens displayed on T7 phage nanoparticles elicits plasma antibody and vaccine-draining lymph node B cell responses. J Immunol Methods 2018; 460:51-62.
9. Shukla G S, Olson W C, Pero S C, et al. Vaccine-draining lymph nodes of cancer patients for generating anti-cancer antibodies. Journal of translational medicine 2017; 15:180.
10. Snyder A, Makarov V, Merghoub T, et al. Genetic basis for clinical response to CTLA-4 blockade in melanoma. N Engl J Med 2014; 371:2189-99.
11. Wang L, Lawrence M S, Wan Y, et al. SF3B1 and other novel cancer genes in chronic lymphocytic leukemia. N Engl J Med 2011; 365:2497-506.
12. Koboldt D C, Larson D E, Wilson R K. Using VarScan 2 for Germline Variant Calling and Somatic Mutation Detection. Curr Protoc Bioinformatics 2013; 44:15 4 1-7.
13. Cingolani P, Platts A, Wang le L, et al. A program for annotating and predicting the effects of single nucleotide polymorphisms, SnpEff: SNPs in the genome of Drosophila melanogaster strain w1118; iso-2; iso-3. Fly (Austin) 2012; 6:80-92.
14. McLaren W, Gil L, Hunt S E, et al. The Ensembl Variant Effect Predictor. Genome Biol 2016; 17:122.
15. The UniProt C. UniProt: the universal protein knowledgebase. Nucleic Acids Res 2017; 45:D158-D69.
16. Fleit H B, Wright S D, Unkeless J C. Human neutrophil Fc gamma receptor distribution and structure. Proc Natl Acad Sci USA 1982; 79:3275-9.
17. Patel Kashyap R. R J T, Barb Adam W. Multiple Variables at the Leukocyte Cell Surface Impact Fc γ Receptor-Dependent Mechanisms. Frontiers in immunology 2019; 10:223.
18. Penfold P L, Walker L C, Roitt I M. Complex arming in antibody-dependent cell-mediated cytotoxicity: ultrastructural studies of the interaction between human effector cells armed with aggregated anti-DNP antibody and DNP-coated erythrocytes. Clin Exp Immunol 1978; 31:197-204.
19. Saksela E, Imir T, Makela O. Specifically cytotoxic human and mouse lymphoid cells induced with antibody or antigen-antibody complexes. J Immunol 1975; 115:1488-92.
20. Slamon D J, Leyland-Jones B, Shak S, et al. Use of chemotherapy plus a monoclonal antibody against HER2 for metastatic breast cancer that overexpresses HER2. N Engl J Med 2001; 344:783-92.
21. Mirzaei H R, Rodriguez A, Shepphird J, Brown C E, Badie B. Chimeric Antigen Receptors T Cell Therapy in Solid Tumor: Challenges and Clinical Applications.

Frontiers in immunology 2017; 8:1850.
22. Schuster S J, Bishop M R, Tam C S, et al. Tisagenlecleucel in Adult Relapsed or Refractory Diffuse Large B-Cell Lymphoma. N Engl J Med 2019; 380:45-56.
23. Park J H, Riviere I, Gonen M, et al. Long-Term Follow-up of CD19 CAR Therapy in Acute Lymphoblastic Leukemia. N Engl J Med 2018; 378:449-59.
24. Parrillo J E, Fauci A S. Apparent direct cellular cytotoxicity mediated via cytophilic antibody. Multiple Fc receptor bearing effector cell populations mediating cytophilic antibody induced cytotoxicity. Immunology 1977; 33:839-50.
25. Perlmann P, Perlmann H. Contactual lysis of antibody-coated chicken erythrocytes by purified lymphocytes. Cellular immunology 1970; 1:300-15.
26. Matlung H L, Babes L, Zhao X W, et al. Neutrophils Kill Antibody-Opsonized Cancer Cells by Trogoptosis. Cell Rep 2018; 23:3946-59 e6.
27. Parrillo J E, Fauci A S. Apparent direct cellular cytotoxicity mediated via cytophilic antibody. Multiple Fe receptor bearing effector cell populations mediating cytophilic antibody induced cytotoxicity. Immunology 1977; 33:839-50.
28. Perlmann P, Perlmann H. Contractual lysis of antibody-coated chicken erythrocytes by purified lymphocytes. Cell Immunol. 1790; 1:300-15.
29. Matlung H L, Babes L, Zhao X W, et al. Neutrophils kill antibody-opsonized cancer cells by trogoptosis. Cell Rep. 2018; 23(3946-59):e6.

### OTHER EMBODIMENTS

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain embodiments of the invention or aspects of the invention consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present invention, as defined in the following claims.

1.-97. (canceled)

98. A method of treating a cancer in a subject, the method comprising administering to a subject having cancer, binding proteins that specifically bind to two or more mutated peptides in the subject in an effective amount to treat the cancer, wherein the two or more mutated peptides each comprise at least one tumor-specific amino acid substitution in an extracellular domain of a membrane protein and/or in a tumor-secreted protein in the subject, and wherein the mutated peptides are not expressed or are expressed in low levels in noncancerous tissues in the subject.

99. The method according to claim 98, wherein the binding proteins specifically bind to at least three, at least four, at least five, at least six, at least seven, at least eight, at least nine, or at least ten of said mutated peptides.

100. The method of claim 98, wherein the administration of the binding proteins that specifically bind to the two or more mutated peptides results in a significant reduction in tumor volume.

101. The method of claim 98, wherein the administration of the binding proteins that specifically bind to the two or more mutated peptides results in a significant increase in survival rate.

102. The method of claim 98, wherein the cancer is selected from the group consisting of basal cell carcinoma, bladder cancer, bone cancer, bowel carcinoma, breast cancer, carcinoid, anal squamous cell carcinoma, castration-resistant prostate cancer (CRPC), cervical carcinoma, colorectal cancer (CRC), colon cancer cutaneous squamous cell carcinoma, endometrial cancer, esophageal cancer, gastric carcinoma, gastroesophageal junction cancer, glioblastoma/mixed glioma, glioma, head and neck cancer, hepatocellular carcinoma, hematologic malignancy. liver cancer, lung cancer, melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer, rectal carcinoma, renal cancer, sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, or uterine sarcoma.

103. The method of claim 98, further comprising administering an immune checkpoint inhibitor to the subject having cancer.

104. The method of claim 103, wherein the binding proteins and the immune checkpoint inhibitor are administered simultaneously.

105. The method of claim 103, wherein the binding proteins are administered prior to administration of the immune checkpoint inhibitor.

106. The method of claim 103, wherein the immune checkpoint inhibitor is selected from the group consisting of pembrolizumab, nivolumab, J43, RMP1-14, atezolizumab, ipilimumab, and combinations thereof.

107. The method of claim 98, wherein the binding proteins are antibodies that specifically bind to the two or more mutated peptides.

108. The method according to claim 107, wherein the antibodies are polyclonal antibodies.

109. The method according to claim 107, wherein the antibodies are administered in a composition that comprises effector cells that are armed with the antibodies.

110. The method according to claim 109, wherein the effector cells are syngeneic donor effector cells.

111. A method for treating a cancer in a subject, the method comprising:
(a) identifying two or more mutated peptides in a subject having cancer, wherein the two or more mutated peptides each comprise at least one tumor-specific amino acid substitution in an extracellular domain of a membrane protein and/or in a tumor-secreted protein in the subject, and wherein the mutated peptides are not expressed or are expressed in low levels in noncancerous tissues in the subject;
(b) vaccinating a second subject with the mutated peptides, thereby producing polyclonal antibodies that specifically bind to the two or more mutated peptides; and
(c) administering the polyclonal antibodies to the subject in an amount effective to treat the cancer.

112. The method according to claim 111, wherein the polyclonal antibodies specifically bind to at least three, at least four, at least five, at least six, at least seven, at least eight, at least nine, or at least ten of said mutated peptides.

113. The method of claim 111, wherein the administration of the antibodies that specifically bind to the two or more mutated peptides results in a significant reduction in tumor volume.

114. The method of claim 111, wherein the administration of the antibodies that specifically bind to the two or more mutated peptides results in a significant increase in survival rate.

115. The method of claim 111, wherein the cancer is selected from the group consisting of basal cell carcinoma, bladder cancer, bone cancer, bowel carcinoma, breast cancer, carcinoid, anal squamous cell carcinoma, castration-resistant prostate cancer (CRPC), cervical carcinoma, colorectal cancer (CRC), colon cancer cutaneous squamous cell carcinoma, endometrial cancer, esophageal cancer, gastric carcinoma, gastroesophageal junction cancer, glioblastoma/mixed glioma, glioma, head and neck cancer, hepatocellular carcinoma, hematologic malignancy. liver cancer, lung cancer, melanoma, Merkel cell carcinoma, multiple myeloma, nasopharyngeal cancer, osteosarcoma, ovarian cancer, pancreatic cancer, peritoneal carcinoma, undifferentiated pleomorphic sarcoma, prostate cancer, rectal carcinoma, renal cancer, sarcoma, salivary gland carcinoma, squamous cell carcinoma, stomach cancer, testicular cancer, thymic carcinoma, thymic epithelial tumor, thymoma, thyroid cancer, urogenital cancer, urothelial cancer, uterine carcinoma, or uterine sarcoma.

116. The method of claim 111, further comprising administering an immune checkpoint inhibitor to the subject having cancer.

117. The method of claim 116, wherein the immune checkpoint inhibitor is selected from the group consisting of pembrolizumab, nivolumab, J43, RMP1-14, atezolizumab, ipilimumab, and combinations thereof.
